SEQUENCE LISTING (54 sequences)

SEQ ID NO: 1 - protein, 17 aa, Artificial sequence
  Avian influenza HA epitope
GGGGSPYQGKSSGGGGS

SEQ ID NO: 2 - protein, 9 aa, Escherichia coli
RAMEAPLRQ

SEQ ID NO: 3 - protein, 9 aa, Escherichia coli
REIELEDKF

SEQ ID NO: 4 - protein, 12 aa, Escherichia coli
RGVNVLADAVKV

SEQ ID NO: 5 - protein, 11 aa, Escherichia coli
RVEDALHATRA

SEQ ID NO: 6 - protein, 13 aa, Escherichia coli
RGQNEDQNVGIKV

SEQ ID NO: 7 - protein, 13 aa, Escherichia coli
REMLPVLEAVAKA

SEQ ID NO: 8 - protein, 14 aa, Escherichia coli
RQQIEEATSDYDRE

SEQ ID NO: 9 - protein, 19 aa, Escherichia coli
RAAVEEGVVAGGGVALIRV

SEQ ID NO: 10 - protein, 16 aa, Escherichia coli
RQQIEEATSDYDREKL

SEQ ID NO: 11 - protein, 20 aa, Escherichia coli
KDTTTIIDGVGEEAAIQGRV

SEQ ID NO: 12 - protein, 20 aa, Escherichia coli
RQIVLNCGEEPSVVANTVKG

SEQ ID NO: 13 - protein, 19 aa, Escherichia coli
REIELEDKFENMGAQMVKE

SEQ ID NO: 14 - protein, 27 aa, Escherichia coli
KANDAAGDGTTTATVLAQAIITEGLKA

SEQ ID NO: 15 - protein, 27 aa, Escherichia coli
KVGKEGVITVEDGTGLQDELDVVEGRQ

SEQ ID NO: 16 - protein, 28 aa, Escherichia coli
KAGKPLLIIAEDVEGEALATLVVNTMRG

SEQ ID NO: 17 - protein, 30 aa, Escherichia coli
KGGDGNYGYNAATEEYGNMIDMGILDPTKV

SEQ ID NO: 18 - protein, 30 aa, Escherichia coli
RGYLSPYFINKPETGAVELESPFILLADKK

SEQ ID NO: 19 - protein, 31 aa, Escherichia coli
RGYLSPYFINKPETGAVELESPFILLADKKI

SEQ ID NO: 20 - protein, 9 aa, Escherichia coli
KDQGIDLRN

SEQ ID NO: 21 - protein, 10 aa, Escherichia coli
RIAGLEVKRI

SEQ ID NO: 22 - protein, 10 aa, Escherichia coli
RNDPLAMQRL

SEQ ID NO: 23 - protein, 11 aa, Escherichia coli
RVLENAEGDRT

SEQ ID NO: 24 - protein, 10 aa, Escherichia coli
KFEELVQTRN

SEQ ID NO: 25 - protein, 10 aa, Escherichia coli
RFQDEEVQRD

SEQ ID NO: 26 - protein, 11 aa, Escherichia coli
RKFEELVQTRN

SEQ ID NO: 27 - protein, 12 aa, Escherichia coli
KVAEFFGKEPRK

SEQ ID NO: 28 - protein, 11 aa, Escherichia coli
RRFQDEEVQRD

SEQ ID NO: 29 - protein, 13 aa, Escherichia coli
RNQGDHLLHSTRK

SEQ ID NO: 30 - protein, 13 aa, Escherichia coli
KLESLVEDLVNRS

SEQ ID NO: 31 - protein, 14 aa, Escherichia coli
KASSGLNEDEIQKM

SEQ ID NO: 32 - protein, 13 aa, Escherichia coli
KVAEFFGKEPRKD

SEQ ID NO: 33 - protein, 13 aa, Escherichia coli
RLINYLVEEFKKD

SEQ ID NO: 34 - protein, 14 aa, Escherichia coli
RNQGDHLLHSTRKQ

SEQ ID NO: 35 - protein, 15 aa, Escherichia coli
KMAPPQISAEVLKKM

SEQ ID NO: 36 - protein, 15 aa, Escherichia coli
RAKLESLVEDLVNRS

SEQ ID NO: 37 - protein, 16 aa, Escherichia coli
KIIAADNGDAWVEVKG

SEQ ID NO: 38 - protein, 16 aa, Escherichia coli
RQAVTNPQNTLFAIKR

SEQ ID NO: 39 - protein, 17 aa, Escherichia coli
KSLGQFNLDGINPAPRG

SEQ ID NO: 40 - protein, 18 aa, Escherichia coli
RIINEPTAAALAYGLDKG

SEQ ID NO: 41 - protein, 17 aa, Escherichia coli
RQAVTNPQNTLFAIKRL

SEQ ID NO: 42 - protein, 19 aa, Escherichia coli
KRIINEPTAAALAYGLDKG

SEQ ID NO: 43 - protein, 21 aa, Escherichia coli
RDAEANAEADRKFEELVQTRN

SEQ ID NO: 44 - protein, 24 aa, Escherichia coli
KIIGIDLGTTNSCVAIMDGTTPRV

SEQ ID NO: 45 - protein, 24 aa, Escherichia coli
RTTPSIIAYTQDGETLVGQPAKRQ

SEQ ID NO: 46 - protein, 24 aa, Escherichia coli
RGMPQIEVTFDIDADGILHVSAKD

SEQ ID NO: 47 - protein, 27 aa, Escherichia coli
RKDVNPDEAVAIGAAVQGGVLTGDVKD

SEQ ID NO: 48 - protein, 26 aa, Escherichia coli
KVALQDAGLSVSDIDDVILVGGQTRM

SEQ ID NO: 49 - protein, 26 aa, Escherichia coli
KIELSSAQQTDVNLPYITADATGPKH

SEQ ID NO: 50 - protein, 26 aa, Escherichia coli
MGKIIGIDLGTTNSCVAIMDGTTPRV

SEQ ID NO: 51 - protein, 28 aa, Escherichia coli
KAKIELSSAQQTDVNLPYITADATGPKH

SEQ ID NO: 52 - protein, 28 aa, Escherichia coli
KTAEDYLGEPVTEAVITVPAYFNDAQRQ

SEQ ID NO: 53 - protein, 548 aa, Escherichia coli
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGRQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMGGIGGVM

SEQ ID NO: 54 - protein, 638 aa, Escherichia coli
MGKIIGIDLGTTNSCVAIMDGTTPRVLENAEGDRTTPSIIAYTQDGETLVGQPAKRQAVTNPQNTLFAIKRLIGRRFQDEEVQRDVSIMPFKIIAADNGDAWVEVKGQKMAPPQISAEVLKKMKKTAEDYLGEPVTEAVITVPAYFNDAQRQATKDAGRIAGLEVKRIINEPTAAALAYGLDKGTGNRTIAVYDLGGGTFDISIIEIDEVDGEKTFEVLATNGDTHLGGEDFDSRLINYLVEEFKKDQGIDLRNDPLAMQRLKEAAEKAKIELSSAQQTDVNLPYITADATGPKHMNIKVTRAKLESLVEDLVNRSIEPLKVALQDAGLSVSDIDDVILVGGQTRMPMVQKKVAEFFGKEPRKDVNPDEAVAIGAAVQGGVLTGDVKDVLLLDVTPLSLGIETMGGVMTTLIAKNTTIPTKHSQVFSTAEDNQSAVTIHVLQGERKRAADNKSLGQFNLDGINPAPRGMPQIEVTFDIDADGILHVSAKDKNSGKEQKITIKASSGLNEDEIQKMVRDAEANAEADRKFEELVQTRNQGDHLLHSTRKQVEEAGDKLPADDKTAIESALTALETALKGEDKAAIEAKMQELAQVSQKLMEIAQQQHAQQQTAGADASANNAKDDDVVDAEFEEVKDKK

(19) United States
(12) Patent Application Publication
(10) Pub. No.: US 2012/0164710 A1
(43) Pub. Date: Jun. 28, 2012

(54) METHOD OF PREPARATION OF A BIOLOGICAL PARTICULATE STRUCTURE

(76) Inventors: Yuan Yuan Fan, Indooroopilly (AU); Anton PJ Middelberg, Brookfield (AU)

(21) Appl. No.: 13/380,300

(22) PCT Filed: Jul. 5, 2010
(86) PCT No.: PCT/AU2010/000855

§ 371 Date: Mar. 12, 2012

(30) Foreign Application Priority Data
Jul. 3, 2009 (AU) 2009903119

(51) Int. Cl. C07K 1/18; C07K 14/005; C07K 1/14; C12N 7/02

(52) U.S. Cl. 435/239; 530/412; 530/416

(57) ABSTRACT

The invention provides methods for the preparation of an isolated virus particle or virus-like particle by treating with an agent such that the particles are preferentially in the aqueous phase. The invention also provides methods of preparing a capsomere that is substantially-free of at least one host cell derived chaperone protein by treatment with an agent to selectively separate the capsomere from at least one chaperone protein.

### FIELD OF THE INVENTION

This invention relates to virus particles, virus-like particles and/or capsomeric components thereof. More particularly, this invention relates to a method for preparation of virus particles, virus-like particles and/or virus capsomere structures by selective separation and applications thereof.

### BACKGROUND TO THE INVENTION

Virus-like particles (VLPs) have applications in a number of molecular and biochemical therapies including gene therapy, drug delivery and vaccination and therefore represent a potentially valuable therapeutic tool. It is increasingly important to develop methods for the preparative and large-scale manufacture of VLPs which provide a consistent and high-quality end-product. Expression of virus-like particles invariably yields a complex mixture of particles regardless of the host expression system employed. The particles have the same protein composition and similar physical size, but the quaternary structure varies greatly. Correct VLPs (herein referred to as ‘cVLPs’) invariably co-exist with aggregated VLPs (herein referred to as ‘aVLPs’) and misformed VLPs (herein referred to as ‘mVLPs’). This complex mixture problem arises even for well-optimised VLP processes.

As the components within a mixture of VLPs all have the same protein composition and very similar particle size, their separation is difficult. Nevertheless, separation is essential as the presence of even small amounts of aggregate can render a vaccine product highly reactogenic. For example, aggregates have been associated with severe disease (Wright, Qu et al, 2003) and altered immunogenic response (Braun, Kwee et al 1997).

While methods for the separation of aggregates are known, these methods are complex, costly and yield-reducing. Moreover, additional difficult-to-validate process steps are invariably required, often based on chromatography. For example, International Publication No. WO 00/09671 describes L1-based VLPs from papillomavirus which involves formation of an L1 VLP in yeast, followed by disassembly and then reassembly to improve VLP structure, followed by removal of aggregates by chromatography.

A related problem is the purification of viral capsomeres. In the same way that building a perfect wall requires perfect bricks as a starting point, building a perfect VLP requires perfect capsomeres. After expression in E. coli, capsomeres typically are imperfect. A small percentage of the capsomeres (in some cases less than a 1%) have protein contaminant bound to them, even after stringent purification by two or three orthogonal chromatography steps (e.g. affinity and size exclusion). These contaminants are not easily detected on e.g. SDS-PAGE, as they are present at such a low relative concentration. Also, a single contaminating protein bound to a capsomere can be difficult to detect as the size shift (e.g. from 230 to 290 kDa) is marginal for the resolution of most preparative size-exclusion columns. Thus the problem of co-purifying bound contaminants has been ignored in the literature.

However, even 1% of “bad bricks” can be disastrous in terms of the quality of the built product. For example, for a VLP that requires 72 perfect capsomeres to self assemble, if 1% of capsomeres are imperfect, then the probability of a perfect VLP being built from the mixture is 0.99⁷²=48.5%. Thus, even 1% of imperfect capsomeres will result in a VLP mixture that has more then 50% of imperfect VLPs in it. This result is disastrous from a processing and product perspective, and highlights why so many electron micrographs in the literature (and industry) show heterogeneous VLPs.

### SUMMARY OF THE INVENTION

Despite an increasing requirement for the commercial scale production of biological agents for the inclusion into pharmaceutical preparations, there still remains a need for methodologies that at least address a number of commercial drivers such as cost, time, scalability, yield and the like.

In a broad form, the invention is directed to methods for the preparation of viral structural protein derived molecules and particularly macromolecular assemblies, which are suited for inclusion into pharmaceutical preparations.

The invention is broadly directed to methods of preparing virus particles and/or VLPs with a desired or preferred quaternary structure from a mixture containing assemblies with heterogeneous or undesirable quaternary structures. In general embodiments, the invention is directed to methods of purifying virus particles and/or VLPs with a desired or preferred quaternary structure from a mixture containing assemblies with heterogeneous or undesirable quaternary structures.

In another broad form, the invention relates to methods of preparing or producing of virus particles and/or VLPs with a desirable quaternary structure which obviates the need for costly and time-consuming downstream purification steps.

In yet another broad form, the invention relates to methods of preparing or producing virus capsomere structures from at least one host derived chaperone protein. In particular embodiments of this form, the method does not include size exclusion chromatography.

In a first aspect, the invention provides a method of preparing an isolated virus particle and/or virus-like particle (VLP), wherein said method includes the step of contacting a mixture comprising an isolated virus particle and/or VLP with an agent at a concentration such that the isolated virus particle and/or VLP is preferentially in an aqueous phase.

In a second aspect, the invention provides an isolated virus particle and/or a VLP prepared according to the method of the first aspect.

In a third aspect, the invention provides a pharmaceutical composition comprising an isolated virus particle and/or a VLP of the second aspect.

Preferably, the pharmaceutical composition is an immunotherapeutic composition.

More preferably, the immunotherapeutic composition is a vaccine.

In a fourth aspect, the invention provides a method of eliciting an immune response in an animal, wherein said method includes the step of administering a pharmaceutical composition of the third aspect to said animal, to thereby elicit an immune response in said animal.

In a fifth aspect, the invention provides a method of immunising an animal, including the step of administering a pharmaceutical composition of the third aspect to said animal, to thereby induce immunity in said animal.

In a sixth aspect, the invention provides a method of treating an animal, including the step of administering a pharmaceutical composition of the third aspect to thereby modulate an immune response in said animal to prophylactically or therapeutically treat said animal.

In a seventh aspect, the invention provides a kit for preparing an isolated virus particle and/or a VLP, wherein said kit comprises one or more agents to prepare an isolated virus particle and/or a VLP such that the isolated virus particle and/or VLP is preferentially in an aqueous phase.

Preferably, the agent of any one of the first to seventh aspects is selected from the group consisting of a polymer, a salt and an acid.

In an eighth aspect, the invention provides a method of preparing a capsomere substantially-free of one or more host cell derived chaperone proteins, said method including the step of contacting a mixture comprising a capsomere and at least one host cell derived chaperone protein with an agent such that the capsomere is selectively separated from at least one host cell derived chaperone protein to thereby prepare a capsomere substantially-free of one or more host cell derived chaperone proteins.

Preferably, the agent is selected from the group consisting of an anion exchanger chromatographic material, a cation exchanger chromatographic material, ammonium sulphate, a PEG and combinations thereof.

More preferably, the agent is ammonium sulphate.

In other preferred embodiments, the agent is ammonium sulphate and an anion exchanger chromatographic material. In yet other preferred embodiments, the agent is ammonium sulphate and a cation exchanger chromatographic material. In still further embodiments, the agent is an anionic exchanger chromatographic material and a cation exchanger chromatographic material.

Preferred embodiments of the eighth aspect relate to a method of preparing a capsomere substantially-free of one or more host cell derived chaperone proteins, wherein said method includes any one or a plurality of the following steps of
(a) contacting said mixture with an anion exchanger chromatographic material to selectively separate the capsomere structure from at least one host cell derived chaperone protein;
(b) contacting said mixture with a cation ion -exchange chromatographic material to selectively separate the capsomere from at least one host cell derived chaperone protein; and
(c) contacting said mixture with ammonium sulphate or PEG to selectively separate the capsomere from at least one host cell derived chaperone protein.

Preferably, the method comprises an ordered sequence of steps (c); and (a) or (b).

In preferred embodiments, step (c) utilises ammonium sulphate.

In preferred embodiments, the method of the eighth aspect does not include size exclusion chromatography.

Preferably, the anion exchanger chromatographic material is quaternary amine (Q).

Preferably, the cation exchanger chromatographic material comprises sulphate, phosphate and carboxylate chromatographic materials. More preferably, the cationic exchanger is sulfopropyl.

Preferably, the host cell derived chaperone protein is a heat shock protein. Even more preferably, the host cell derived chaperone is a heat shock protein from bacteria and yet even more preferably, E. coli.

Preferably, the heat shock protein is selected from heat shock protein 60 (Hsp60), heat shock protein 70 (Hsp70), GroEL and dnaK.

In an ninth aspect, the invention provides a capsomere substantially-free of one or more host cell derived chaperone proteins produced according to the method of the eighth aspect.

In a tenth aspect, the invention provides a pharmaceutical composition comprising the capsomere of the ninth aspect.

Preferably, the pharmaceutical composition is an immunotherapeutic composition.

More preferably, the immunotherapeutic composition is a vaccine.

In an eleventh aspect, the invention provides a method of eliciting an immune response in an animal, wherein said method includes the step of administering a pharmaceutical composition of the tenth aspect to said animal, to thereby elicit an immune response in said animal.

In a twelfth aspect, the invention provides a method of immunising an animal, including the step of administering a pharmaceutical composition of the tenth aspect to said animal, to thereby induce immunity in said animal.

In a thirteenth aspect, the invention provides a method of treating an animal, including the step of administering a pharmaceutical composition of the tenth aspect to thereby modulate an immune response in said animal to prophylactically or therapeutically treat said animal.

In a fourteenth aspect, the invention provides a kit preparing a capsomere substantially-free of one or more host cell derived chaperone proteins wherein said kit comprises one or more agents, such that the capsomere is selectively separated from at least one host cell derived chaperone protein to thereby prepare a capsomere substantially-free of one or more host cell derived chaperone proteins.

Preferably, the one or more agents of the fourteenth aspect is selected from the group consisting of selected from the group consisting of an anion exchanger chromatographic material, a cation exchanger chromatographic material, ammonium sulphate, a PEG and combinations thereof.

In preferred embodiments of any one of the aforementioned aspects, the polymer is selected from the group consisting of polyethylene glycol (PEG) and a polyelectrolyte.

More preferably, the PEG has an average molecular weight of between about 1000 Da and about 100000 Da.

Even more preferably, the PEG has an average molecular weight of between about 4500 Da and about 70000 Da.

Yet even more preferably, the PEG molecule has an average molecular weight of between about 5000 Da and about 6000 Da.

In preferred embodiments which relate to PEG, the PEG has a final concentration of between about 1.5% w/v and about 5.5% w/v. More preferably, the PEG has a final concentration of between about 3.75% w/v and about 4.25% w/v.

Even more preferably, the PEG has a final concentration of about 4% w/v.

In other preferred embodiments, the polyelectrolyte is an anionic polyelectrolyte.

Preferably, the salt is selected from ammonium sulphate and sodium chloride. More preferably, the salt is ammonium sulphate.

In preferred embodiments which relate to preparation of an isolated virus particle and/or VLP, the ammonium sulphate has a final concentration of between about 20% v/v of saturated ammonium sulphate and about 40% v/v of saturated ammonium sulphate. More preferably, the concentration of ammonium sulphate is between about 23% v/v of saturated ammonium sulphate and about 35% v/v of saturated ammonium sulphate. Even more preferably, the ammonium sulphate has a final concentration of between about 24% v/v of saturated ammonium sulphate and about 33% v/v of saturated ammonium sulphate. Yet even more preferably, the ammonium sulphate has a final concentration of between about 25% of saturated ammonium sulphate and about 27% v/v of saturated ammonium sulphate.

In preferred embodiments that relate to capsomere preparation, the concentration of ammonium sulphate is at least about 12% v/v of saturated ammonium sulphate. More preferably, the concentration of ammonium sulphate is between about 12% v/v of saturated ammonium sulphate and about 50% v/v of saturated ammonium sulphate. Even more preferably, the concentration of ammonium sulphate is between about 12% of saturated ammonium sulphate and 30% v/v of saturated ammonium sulphate. Yet even more preferably, the concentration is between about 15% v/v and about 25% v/v of saturated ammonium sulphate. In particularly preferred embodiments, the final concentration of ammonium sulphate is about 20% v/v of saturated ammonium sulphate.

Preferably, the acid is phosphoric acid.

In preferred embodiments, the isolated virus particle, capsomere and/or the VLP according to any one of the aforementioned aspects comprise one or more isolated proteins comprising a virus capsid protein amino acid sequence.

Preferably, the virus capsid protein comprises polyomavirus VP1 amino acid sequence and even more preferably a murine polyomavirus VP1 amino acid sequence. In particularly preferred embodiments, the VLP comprises one or more isolated proteins comprising a polyomavirus VP1 amino acid sequence and more preferably, a murine polyomavirus VP1 amino acid sequence.

In other preferred embodiments, the VLP according to any one of the aforementioned aspects comprises one or more isolated proteins comprising a human papillomavirus (HPV) capsid protein amino acid sequence. More preferably, the HPV capsid protein amino acid sequence is selected from L1 and L2. Even more preferably, the HPV capsid protein amino acid sequence is L1.

Preferably, the virus capsid protein amino acid sequence further comprises one or more immunogenic epitopes of a pathogen other than said virus.

In preferred embodiments, the pathogen is selected from influenza virus, Hendra virus and Group A Streptococcus pyogenes. More preferably, the pathogen is influenza virus.

In particularly preferred embodiments of any one of the aforementioned aspects, when the VLP comprises one or more isolated proteins comprising a polyomavirus VP1 amino acid sequence in the absence of one or more immunogenic epitopes, preferably the concentration of ammonium sulphate is between about 25% v/v and about 32% v/v and more preferably, about 32% v/v.

In other particularly preferred embodiments, when the VLP comprises one or more isolated proteins comprising a polyomavirus VP1 amino acid sequence further comprises one or more immunogenic epitopes derived from influenza virus, the concentration of ammonium sulphate is preferably between about 24% v/v and 33% v/v of saturated ammonium sulphate and yet even more preferably, about 25% v/v of saturated ammonium sulphate.

In preferred embodiments of that relate to a kit, the invention provides a kit when used for preparing an isolated virus particle,r a VLP and/or a capsomere.

Preferably, the animal of any one of the aforementioned aspects is a mammal and more preferably, the mammal is a human.

Although the invention is preferably directed to humans, it will be appreciated that the invention is also applicable to other mammals such as livestock, performance animals, domestic pets and the like.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF FIGURES

In order that the invention may be readily understood and put into practical effect, preferred embodiments will now be described by way of example with reference to the accompanying:

FIG. 1 Effect of (NH₄)₂SO₄ concentration (% v/v) on the size distribution of wild-type VP1 virus-like particles remaining in the solution supernatant (SN). Y axis is normalised UV absorbance; X axis is time in minutes. — is untreated control; [symbol] is SN (12.5%); ---- is SN (20%); —••— is SN (27%); ——— is SN (33%).

FIG. 2 Effect of (NH₄)₂SO₄ concentration (% v/v) on the size distribution of wild-type VP1 virus-like particles remaining in the solution supernatant (SN). Y axis is UV absorbance normalised to control; X axis is time in minutes. — is untreated control; [symbol] is SN (28%); - - - - is SN (30%); —••— is SN (32%).

FIG. 3 Effect of (NH₄)₂SO₄ concentration (% v/v) on the size distribution of wild-type VP1 virus-like particles partitioning to the precipitate (PPT). — is untreated control; [symbol] is PPT (28%); ----- is PPT (30%); —••— is PPT (32%). Y axis is UV absorbance normalised to control; X axis is time in minutes.

FIG. 4 Effect of precipitation with 32% v/v saturated (NH₄)₂SO₄ on virus-like particle quality. A comparison of particles partitioning to the precipitate (PPT) and supernatant (SN). — is untreated control; [symbol] is PPT; - - - - is SN. Y axis is UV absorbance normalised to control; X axis is time in minutes.

FIG. 5 Effect of PEG concentration (% w/v) on the size distribution of wild-type VP1 virus-like particles remaining in the solution supernatant (SN). — is untreated control; [symbol] is SN (2%); - - - - is SN (4%); —•6108 — is SN (6%). Y axis is UV absorbance normalised to control; X axis is time in minutes.

FIG. 6 Effect of PEG concentration (% w/v) on the size distribution of wild-type VP1 virus-like particles remaining in the solution supernatant (SN). — is untreated control; [symbol] is SN (3%); - - - - is SN (4%); —••— is SN (5%). Y axis is UV absorbance normalised to control; X axis is time in minutes.

FIG. 7 Effect of precipitation with 4% w/v PEG on virus-like particle quality. A comparison of particles partitioning to the precipitate (PPT) and supernatant (SN). — is untreated control; [symbol] is PPT; - - - - is SN. Y axis is UV absorbance normalised to control; X axis is time in minutes.

FIG. 8 Transmission electron micrographs at 200 000× magnification of samples from 32% (NH₄)₂SO₄ treatment. A is control sample; B is 32% ammonium sulphate (AS) supernatant sample; C. 32% AS precipitate sample. Bar is 100 nm=1.1 cm.

FIG. 9 Transmission electron micrographs at 200 000× magnification of samples from 4% PEG treatment. A is control sample; B is 4% PEG SN; C is 4% PEG PPT. Bar is 1.3 cm=100 nm.

FIG. 10 Electron micrographs at 100 000× magnification of chimeric VLPs remaining in the solution of supernatant (SN) after ammonium sulphate treatment. A. Control; B. 23% AS SN; C. 25% AS SN; D. 27% AS SN; E. 29% AS SN; F. 31% AS SN. The white bar present on each micrograph is 1.4 cm which is equivalent to 200 nm.

FIG. 11 Size exclusion (S200) chromatography to purify VP1 after thrombin treatment. A is the chromatograph of the entire column run whilst B. is an expanded view of the capsomere peak.

FIG. 12 SDS-PAGE analysis of GroEL complex (referred to as ‘GroEL’) and dnaK proteins in different fractions from S200 chromatography Lane 1: Protein marker, Lane 2: GST VP1 mixture digested with thrombin, Lane 3: Fraction A3 from S200 chromatography, Lane 4: Fraction A5 from S200 chromatography, Lane 5: Fraction A8 from S200 chromatography, Lane 6: Fraction A12 from S200 chromatography, Lane 7: Fraction B10 from S200 chromatography. The upper arrow indicates dnaK; the lower arrow is GroEL. The bands indicated with a box and asterisk were sent for peptide mass fingerprinting.

FIG. 13 Size exclusion (S200) chromatography to purify VP1 after thrombin treatment. Effect of GroEL and dnaK proteins on VP1 assembly using different S200 fractions. Y axis is UV absorbance at 280 nm while X-axis is Time (min). [symbol] is Fraction B10; - - - - is Fraction A12; —is Fraction A8; ———— is Fraction A3; —••—••— is Fraction A5.

FIG. 14 MALDI-MS data for GroEL bands on FIG. 12.

FIG. 15 Probability Based Mowse Score for GroEL bands on FIG. 12.

FIG. 16 Identified match sequence (Q6UDB4) for GroEL bands.

FIG. 17 MALDI-MS data for dnaK bands on FIG. 12.

FIG. 18 Probability Based Mowse Score for dnaK bands on FIG. 12.

FIG. 19 Identified match sequence (A7ZHA4) for dnaK bands.

FIG. 20 SDS-PAGE analysis of dnaK protein in different (NH₄)₂SO₄ solutions. The arrow indicates a band corresponding to dnaK. Lane 1. Protein marker; Lane 2. VP1 purified from S200 chromatography (S200VP1); Lane 3. VP1 supernatant after treated with (NH₄)₂SO₄ at Conc. 12.5%; Lane 4. VP1 resuspension after treated with (NH₄)₂SO₄ at Conc. 12.5% (2× concentrated). Lane 5. VP1 supernatant after treated with (NH₄)₂SO₄ at Conc. 20%; Lane 6. VP1 resuspension after treated with (NH₄)₂SO₄ at Conc. 20%; Lane 7. VP1 supernatant after treated with (NH₄)₂SO₄ at Conc.25%; Lane 8. VP1 resuspension after treatment with (NH₄)₂SO₄ at Conc. 25%; Lane 9. VP1 supernatant after treated with (NH₄)₂SO₄ at Conc. 30%; Lane 10. VP1 resuspension after treated with (NH₄)₂SO₄ at Conc. 30%.

FIG. 21 Electron micrograph of (NH₄)₂SO₄ purified capsomere and assembled VLP. A is S200 VP1 resuspension after treated with (NH₄)₂SO₄ at Conc. 25%; B is VLP (assembled using (NH₄)₂SO₄ purified S200VP1); C is VLP (assembled using S200VP1 without (NH₄)₂SO₄ treatment).

FIG. 22 SDS-PAGE analysis of dnaK protein in different (NH₄)₂SO₄ solutions. Lane 1. Protein marker; Lane 2. VP1 purified from QFF chromatography (QFFVP1); Lane 3. VP1 supernatant after treated with (NH₄)₂SO₄ at Conc. 25%; Lane 4. VP1 resuspension after treated with (NH₄)₂SO₄ at Conc. 25% (2× concentrated); Lane 5. VP1 supernatant after treated with (NH₄)₂SO₄ at Conc. 50%; Lane 6. VP1 resuspension after treated with (NH₄)₂SO₄ at Conc. 50% (2× concentrated).

FIG. 23 Electron micrograph of (NH₄)₂SO₄ purified capsomere. A is QFFVP1 capsomeres (without (NH₄)₂SO₄ treatment); B is QFFVP1 resuspension after treated with (NH₄)₂SO₄ at Conc. 25%.

FIG. 24 Ion Exchange (IEX) chromatography using QFF column to purify VP1 after thrombin treatment. Y axis is UV absorbance at 280 nm; X axis is volume (ml).

FIG. 25 SDS-PAGE analysis of GroEL and dnaK proteins in different fractions from ion-exchange chromatography using QFF column. Lane 1. GST VP1 mixture digested with thrombin; Lane 2. Fraction A5 from binding step of QFF chromatography; Lane 3. Fraction B5 from GST eluting step of QFF chromatography Lane 4. Fraction D11 from VP1 eluting step of QFF chromatography; Lane 5. Pooled fractions D9 and D10 from VP1 eluting step of QFF chromatography; Lane 6. Protein marker.

FIG. 26 IEX chromatography to purify VP1 after thrombin treatment. A. QFF chromatography (step 1). B. SP chromatography (step 2).

FIG. 27 SDS-PAGE analysis of different fractions from TEX chromatography. Lane 1. Protein marker; Lane 2. QFFVP1 purified from QFF chromatography; Lane 3. Fraction AS from binding step of SP chromatography; Lane 4. Fraction A7 from binding step of SP chromatography; Lane 5. Fraction A10 from washing step of SP chromatography; Lane 6. Fraction A12 from washing step of SP chromatography; Lane 7. Fraction B4 purified from QFF and SP chromatography; Lane 8. Fraction B3 purified from QFF and SP chromatography.

FIG. 28 Electron micrograph of capsomere purified from IEX chromatography and assembled VLP. A. VP1 purified by 2-step IEX chromatography; B. VLP.

FIG. 29 SDS-PAGE analysis of dnaK and GroEL protein in different (NH₄)₂SO₄ solutions. Lane 1. Protein marker; Lane 2. VP1 sample 1 purified by S200 chromatography; Lane 3. VP1 sample 1 supernatant after treatment with (NH₄)₂SO₄ at 15% (v/v); Lane 4. VP1 sample 1 resuspension after treatment with (NH₄)₂SO₄ at 15% (v/v); Lane 5. VP1 sample 1 supernatant after treatment with (NH₄)₂SO₄ at 20% (v/v); Lane 6. VP1 sample 1 resuspension after treatment with (NH₄)₂SO₄ at 20% (v/v); Lane 7. VP1 sample 2 purified by S200 chromatography; Lane 8. VP1 sample 2 supernatant after treatment with (NH₄)₂SO₄ at 15% (v/v); Lane 9. VP1 sample 2 resuspension after treatment with (NH₄)₂SO₄ at 15% (v/v); Lane 10. VP1 sample 2 supernatant after treatment with (NH₄)₂SO₄ at 20% (v/v); Lane 11. VP1 sample 2 resuspension after treatment with (NH₄)₂SO₄ at 20% (v/v); Lane 12. VP1 GST mixture following digestion with thrombin; Lane 13. VP1 GST mixture supernatant after treatment with (NH₄)₂SO₄ at 15% (v/v); Lane 14. VP1 GST mixture resuspension after treatment with (NH₄)₂SO₄ at 15% (v/v); Lane 15. VP1 GST mixture supernatant after treatment with (NH₄)₂SO₄ at 20% (v/v); Lane 16. VP1 GST mixture resuspension after treatment with (NH₄)₂SO₄ at 20% (v/v).

FIG. 30 Electron micrograph of assembled VLPs from FIG. 29 (the scale bar 1.5 cm=200 nm) Panel A. VLP (assembled using S200 VP1 sample 1 without (NH₄)₂SO₄ treatment); Panel B. VLP (assembled using S200 VP1 sample 1 after treatment with (NH₄)₂SO₄ at Conc. 15%); Panel C. VLP (assembled using VP1 and GST mixture without (NH₄)₂SO₄ treatment); Panel D. VLP (assembled using VP1 and GST mixture purified with 15% (NH₄)₂SO₄ treatment.

### DETAILED DESCRIPTION OF THE INVENTION

The present invention is predicated, at least in part, on the finding that VLPs with a desired or correct quaternary structure may be selectively separated from viral structural protein assemblies which are either aggregated and/or structurally misformed. Moreover, the inventors have surprisingly found that treatment of VLPs with agents that precipitate proteins at certain concentrations retains the correctly formed VLPs to remain in solution without damaging the structural integrity of the correctly formed VLP. The misformed virus particles or VLPs remain as aggregates in the precipitate. As will be appreciated, the method of the present invention provides any one or a plurality of advantages over conventional processes for the production of VLPs inclusive of (i) rapid bench scale optimisation in kit format; (ii) scalable for clinical trial and market supply; (iii) lower cost; (iv) simple; (v) high yield of VLPs of interest and/or (v) obviates the requirement for complex and costly purification steps.

The present inventors have also devised methodologies to address the problem of capsomere purification. The expression of capsomeres for the preparation of virus-like particles invariably yields a complex mixture of capsomeres with and without bound contaminants. However, some contaminants which have potentially deletrious results on VLP assembly, are present at very low levels (estimated around 1%). However, even 1% of bound contaminant can cause the yield of structurally correct VLPs to be less than 50%. Additionally, the conventional process routes based on SEC (which anyway do not remove these 1% of problem capsomeres) are inefficient and costly to scale. The methods of the present invention are particularly amenable for large- or industrial-scale preparation of capsomeres, virus particles and/or VLPs.

Therefore in broad aspects, the invention relates to methods of preparing an isolated virus particle, a VLP and/or a capsomere by treatment steps that selectively separate these preferred molecules from co-purifying protein contaminants and/or misformed virus particles. In particular embodiments, the methods of invention are methods of purifying an isolated virus particle, a VLP and/or a capsomere.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

As used herein, by “synthetic” is meant not naturally occurring but made through human technical intervention. In the context of synthetic proteins and nucleic acids, this encompasses molecules produced by recombinant, chemical synthetic or combinatorial techniques as are well understood in the art.

By “protein” is meant an amino acid polymer. The amino acids may be natural or non-natural amino acids, D- or L-amino acids or chemically-derivatized amino acids as are well understood in the art.

A “polypeptide” is a protein having fifty (50) or more amino acids.

A “peptide” is a protein having less than fifty (50) amino acids.

Proteins and peptides may be useful in native, chemical synthetic or recombinant synthetic form and may be produced by any means known in the art, including but not limited to, chemical synthesis, recombinant DNA technology and proteolytic cleavage to produce peptide fragments.

In one embodiment, proteins of the invention are produced by chemical synthesis. Chemical synthesis techniques are well known in the art, although the skilled person may refer to Chapter 18 of CURRENT PROTOCOLS IN PROTEIN SCIENCE Eds. Coligan et. al., John Wiley & Sons NY (1995-2009) for examples of suitable methodology.

In another embodiment, proteins may be prepared as a recombinant protein.

The term “recombinant” as used herein refers to a molecule resulting from in vitro manipulation into a form not normally found in nature.

A recombinant protein or peptide may be conveniently prepared by a person skilled in the art using standard protocols as for example described in Sambrook et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-2009), incorporated herein by reference, in particular Chapters 10 and 16; and CURRENT PROTOCOLS IN PROTEIN SCIENCE Eds. Coligan et al., (John Wiley & Sons, Inc. 1995-2009) which is incorporated by reference herein, in particular Chapters 1, 5 and 6.

By “purify”, “purified” and “purification”, particularly in the context of recombinant protein purification, is meant enrichment of a protein and preferably a recombinant protein so that the relative abundance and/or specific activity of said protein and preferably recombinant protein is increased compared to that before enrichment. In preferred embodiments, “purity” relates to at least 60%, 65%, 70%, 75%, 80%, 85% and more preferably 90%, 95%, 96%, 98%, 99% and 100% purity of a desired molecule.

In those embodiments which contemplate peptides, said peptides may be in the form of peptides prepared by chemical synthesis, inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al., (John Wiley & Sons, Inc. NY USA 1995-2009). In this regard, reference is also made to International Publication WO 99/02550 and International Publication WO 97/45444. Reference is also made to International Publication WO2008/040060, which describes expression protocols for VLP and/or capsomere production and in particular, expression of polyomavirus VP1 for self-assembly, and in particular a chimeric VP1 protein comprising peptides or epitopes from a virus protein other than VP1, which may be of particular relevance to the present invention.

The present invention also extends to use of fragments. In one embodiment, a “fragment” includes a protein comprising an amino acid sequence that constitutes less than 100% of an amino acid sequence of an entire protein. A fragment preferably comprises less than 99%, 98%, 97%, 96%, 95%, 94%, 93%, 92%, 91%, 90%, 85%, 80%, 75%, 70%, 60%, 50%, 40%, 30%, 20% or as little as even 10%, 5% or 3% of the entire protein.

The fragment may include a “biologically-active fragment” has no less than 10%, preferably no less than 25%, more preferably no less than 50% and even more preferably no less than 75, 80, 85, 90 or 95% of a biological activity of a protein from which it is derived. In another embodiment, said “biologically-active fragment” has no less than 10%, preferably no less than 25%, more preferably no less than 50% and even more preferably no less than 75, 80, 85, 90 or 95% of a contiguous amino acid sequence of a protein from which it is derived.

By way of example, a “biologically-active fragment” may be a fragment of virus structural protein which retains the ability to self-assemble into a VLP or virus particle.

The terms “contacting”, “contact” or “contacted” includes to treat, subject or otherwise expose.

Particular aspects of the present invention relate to methods of preparing virus particles or VLPs by employing an agent under such conditions such that the virus particle or VLP is preferentially in an aqueous phase whilst misformed or aggregated virus particles or VLPs are precipitated or remain in an insoluble form. In particular embodiments, the invention relates to methods for preparing an isolated virus particle and/or VLP containing preparation that is substantially-free or essentially-free of misformed or aggregated virus particles and/or VLPs.

By “viral particle”, “virus” or “virus particle” is meant a native molecule comprising one or a plurality of virus structural proteins (or fragments or portions thereof) wherein the native molecule is produced by replication of native viral genetic material. It will be understood that a “viral particle” is isolated from nature and is a product of native viral replication. A “virus particle” may optionally include viral genetic material and more particularly, infectious viral genetic material and thereby includes within its scope a virion. A virus particle may also include a lipid envelope. Virus particles may be derived or isolated from tissue culture propagation techniques using conventional methods as are known in the art or alternatively, may be derived or isolated from a cell, tissue, organ, plasma or blood of an infected animal.

In the context of the present invention, by “virus-like particle” or “VLP” is meant a molecule not normally found in nature which comprises one or a plurality of virus structural proteins (or fragments or portions thereof) assembled into a molecule which has a quaternary structure that mimics or resembles the overall structure of a corresponding wild-type, native and/or authentic virus particle. Therefore it will be appreciated that a “VLP” is morphologically similar to a corresponding authentic, wild-type and/or native virus particle since the VLP has an authentic conformation of viral structural proteins. A VLP may be engineered or a product of recombinant technology and more particularly recombinant DNA technology, although without limitation thereto. The one or a plurality of virus structural proteins which form a VLP includes any structural protein amino acid sequence of a virus which can form part of a virus particle structure and is inclusive of a capsid protein and an envelope protein. A VLP of the present invention may comprise a single species of structural protein such as a single capsid protein or alternatively, multiple structural proteins. VLPs may include individual structural proteins, i.e., protein monomers, or dimers, or protein complexes spontaneously formed upon purification of recombinant structural proteins, i.e., self-assembling or intact VLPs. VLPs may also be in the form of capsid monomers, protein or peptide fragments of VLPs or capsid monomers, or mixtures thereof. It is further contemplated that a VLP may further optionally comprise a lipid envelope and/or an isolated genetic material such as DNA and/or RNA. It is envisaged that a VLP may be produced using structural protein fragments or mutated forms thereof, e.g., structural proteins that have been modified by the addition, substitution or deletion of one or more amino acids, although without limitation thereto.

In particular embodiments, the invention may relate to selective separation of isolated virus particle and/or VLPs in an aqueous phase or soluble form. By “selectively separate” or “selective separation” is meant a preferential separation, segregation, enrichment, partitioning, removal or division of an isolated virus particle and/or VLP with a correctly formed structure in an aqueous phase (or other soluble form) with the simultaneous enrichment of aggregated and misformed virus particles and/or VLPs in the precipitate after treatment with an agent. Therefore a preparation comprising an isolated virus particle and/or VLP prepared according to the methods of the present invention preferably comprises at least 65%, 70%, 75%, 80%, 85% and more preferably 90%, 95%, 96%, 98%, 99% and 100% of an isolated virus particle and/or VLP, as measured by isolated virus particle or VLP quality methods as are known in the art.

The term “capsomere” is well-known in the art as a morphological unit of the capsid of a virus. A “capsomere” comprises monomeric or oligomeric viral structural proteins. The capsomeres self-assemble into a virus-like particle. By way of example for VP1 VLPs, five VP1 units self-associate to create a basic capsomeric unit. The VP1 VLP comprises 72 capsomeres associated into a capsid.

In particular aspects, the invention provides to methods for preparation of “a capsomere substantially-free of one or more host cell derived chaperone protein” which in the context of the present invention relates to a capsomere which having undergone the treatment steps of the invention, is not bound to or in complex with one or more host cell derived chaperone proteins which typically co-purifies with said capsomere. By substantially-free it is meant that the capsomere is essentially-free of co-purifying chaperone proteins such that the level chaperone protein is preferably less than 5%, more preferably less than 2.5%, 1%, 0.5% and even less than 0.2% of the total protein content. In preferred embodiments, the one or more chaperone proteins is a heat shock protein, more preferably a heat shock protein from E. coli and yet even more preferably, selected from dnaK and GroEL.

The capsomeres prepared by the methods of the present invention have a superior ability to self-assemble into structurally authentic VLPs when compared to capsomeres with co-bound chaperone proteins. In particular embodiments, capsomeres of the present invention are prepared such that there is a sufficient purity to form VLPs wherein at least 90%, 95%, 97%, 99% or 100% of the resultant VLPs have the correct structure.

By “selectively separate from at least one host cell derived chaperone protein” in the context of a capsomere, is meant a preferential separation, segregation, enrichment, release, removal or partitioning of a capsomere from co-purifying chaperones and preferably, co-purifying chaperone proteins which are bound to or are in complex with a capsomere. In one particular embodiment, to “selectively separate” is meant to selectively release a chaperone protein from a capsomere-chaperone complex. Therefore a preparation comprising an isolated virus particle and/or VLP prepared according to the methods of the present invention preferably comprises at least 65%, 70%, 75%, 80%, 85% and more preferably 90%, 95%, 96%, 98%, 99% and 100% of an isolated virus particle and/or VLP,

Chaperone proteins are well known to a person of skill in the art. Chaperones are wide family of proteins that are found in eukaryotic and prokaryotic cells and function to assist the non-covalent folding or unfolding and the assembly or disassembly of other macromolecular structures, but do not occur in these structures when the latter are performing their normal biological functions. Many chaperones are heat shock proteins, that is, proteins expressed in response to elevated temperatures or other cellular stresses; addition of the prefix “Hsp” for heat shock protein is well known in the art however certain heat shock proteins.

In preferred embodiments of the present invention, the at least one host cell derived chaperone protein is a heat shock protein. More preferably, the heat shock protein is selected from GroEL and dnaK. In other preferred embodiments, the heat shock protein is a chaperone protein from E. coli, and more preferably an E. coli chaperone protein selected from GroEL and dnaK.

It will be understood that “a mixture comprising an isolated virus particle and/or a VLP” is any solution or other type of preparation which comprises an isolated virus particle and/or a VLP with a correct or desirable tertiary or quaternary structure and one or more molecules other than said isolated virus particle and/or said VLP, which comprise a viral structural protein amino acid sequence which have incorrect or undesirable structural properties. In particular embodiments, the mixture consists essentially of an isolated virus particle and/or a VLP, which is a mixture that is substantially devoid of other protein impurities and microcontaminants and typically has undergone an enrichment and/or purification step prior to treatment with an agent in order to remove such impurities and microcontaminants.

It will be appreciated that in preferred embodiments of the invention that relate to capsomere, a mixture consists essentially of a capsomere and at least one host cell derived chaperone protein.

The mixture as used in any aspect of the present invention can be prepared or produced by any number of methods which are suitable for the preparation of virus particles, VLPs or capsomeres as are well known in the art. Capsomeres and VLPs may be produced in vitro and in vivo, in suitable host cells, e.g., mammalian, yeast, bacterial, and insect host cells inclusive of cells capable of producing VLPs, or in an appropriate animal host. Suitable host cells for expression include Escherichia coli (BL21 and DH5α for example), yeast cells (Pichia pastoris, Saccharomyces cerevisiae for example), Sf9 cells utilized with a baculovirus expression system, CHO cells, COS, CV-1, NIH 3T3 and 293 cells, although without limitation thereto. In preferred embodiments, the host cell is E. coli.

According to these embodiments, the mixture may be a cell lysate or cell culture supernatant prepared from a host cell line. Alternatively, the cell lysate or cell culture supernatant may be subjected to one or more processing or purification steps preceding the step of contacting with an agent to yield said mixture.

A mixture comprising a VLP may also be a product of an assembly reaction in which a virus structural protein has been recombinantly expressed and is assembled into a VLP under conditions which promote self-assembly of the virus structural protein into VLPs. According to these embodiments, the virus structural protein which undergoes assembly can be a monomer or alternatively, a multimer.

In alternative embodiments, a VLP may be produced by introduction into a host cell, tissue or organ, of an isolated nucleic acid encoding a substantially entire genome of a virus (such as an infectious clone of a virus) which has been engineered to initiate and maintain viral replication and assembly.

It will be appreciated that in any method of the present invention, the mixture comprising a capsomere, virus particle and/or VLP may be partially purified prior to the treatment steps of the present invention using other purification methods as discussed above. In certain preferred embodiments that utilise a recombinant protein fused to an affinity tag, affinity chromatography is employed prior to the treatment methods of the invention. In particularly preferred embodiments, the affinity chromatography is designed to remove free GST tag. In other certain preferred embodiments, size-exclusion chromatography is used to partially purify a mixture prior to treatment methods of the present invention.

In certain preferred embodiments that relate to capsomere preparation, the mixture comprising a capsomere and at least one chaperone has not undergone size exclusion chromatography prior to the treatment methods.

The phrase “one or more molecules other than said isolated virus particle and/or said VLP, which comprise a viral structural protein amino acid sequence” refers to molecules comprising virus structural proteins with an incorrect tertiary structure (otherwise referred to as ‘misfolded virus structural proteins’). In particular embodiments, said molecules are protein aggregates or misformed proteins. Said molecules may be monomers or multimeric assemblies.

It will be appreciated that an agent as used in the present invention may in certain embodiments, be a protein precipitating agent.

A skilled addressee will appreciate that according to methods which relate to preparation of an isolated virus particle and/or VLP, contact of a mixture with an agent selected from a polymer, a salt and an acid is such that the isolated virus particle and/or VLP is preferentially in a soluble form in the aqueous phase whilst misformed or aggregated virus particles and/or VLPs are in insoluble form.

In certain embodiments of any one of the methods of the present invention, the agent is a polymer and in particular, a PEG. PEG may also have a branched or unbranched structure.

The PEG molecule suited for use in the present invention may be of any molecular weight between about 1 kDa to about 100 kDa, as practically desired. Typically, although not exclusively, PEG preparations exist as a heterogeneous mixture of PEG molecules either above or below the stated molecular weight. By way of example, the PEG may have an average molecular weight of about 1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500, 20000, 25000, 30000, 35000, 40000, 45000, 50000, 55000, 60000, 65000, 70000, 75000, 80000, 85000, 90000, 95000 or 100000 Da.

Preferably, the PEG has an average molecular weight of about between about 4500 Da to about 70000 Da.

More preferably, the PEG has an average molecular weight of about 5500 Da.

Preferably, the PEG is present at concentration of about 1.5% w/v, about 1.6% w/v, about 1.7% w/v, about 1.8% w/v, about 1.9% w/v, about 2.0% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3.0% w/v, 3.05% w/v, about 3.1% w/v, about 3.15% w/v, about 3.2% w/v, about 3.25% w/v, about 3.3% w/v, about 3.35% w/v, about 3.4% w/v, about 3.45% w/v, about 3.5% w/v, about 3.55% w/v, about 3.6% w/v, about 3.65% w/v, about 3.7% w/v, about 3.75% w/v, about 3.8% w/v, about 3.85% w/v, about 3.9% w/v, about 3.95% w/v, about 4.0% w/v, about 4.05% w/v, about 4.1% w/v, about 4.15% w/v, about 4.2% w/v, about 4.25% w/v, about 4.3% w/v, about 4.35% w/v, about 4.4% w/v, about 4.45% w/v, about 4.5% w/v, about 4.55% w/v, about 4.6% w/v, about 4.7% w/v, about 4.8% w/v, about 4.9% w/v, about 5.0% w/v, about 5.1% w/v, about 5.2% w/v, about 5.3% w/v, about 5.4% w/v and about 5.5% w/v.

In other particular embodiments, the polymer is a polyelectrolyte. Preferably the polyelectrolyte is an anionic polyelectrolyte. The anionic polyelectrolyte may be selected from the group consisting of polystyrenesulfonic acid, polyacrylic acid and polystyrenesulfonic acid.

In embodiments which contemplate addition a salt, preferably the salt is selected from ammonium sulphate and sodium chloride. Preferably the salt is ammonium sulphate.

In preferred embodiments which relate to preparation of an isolated virus particle and/or VLP, the ammonium sulphate has a final concentration of between about 20% v/v of saturated ammonium sulphate and about 40% v/v of saturated ammonium sulphate. More preferably, the concentration of ammonium sulphate is between about 23% v/v of saturated ammonium sulphate and about 35% v/v of saturated ammonium sulphate. Even more preferably, the ammonium sulphate has a final concentration of between about 24% v/v of saturated ammonium sulphate and about 33% v/v of saturated ammonium sulphate.

In certain preferred embodiments, the ammonium sulphate has a final concentration of between about 25% of saturated ammonium sulphate and about 27% v/v of saturated ammonium sulphate and even more preferably, about 25% v/v of saturated ammonium sulphate.

In other preferred embodiments, the ammonium sulphate has a final concentration of between about 27% v/v and about 33% v/v of saturated ammonium sulphate and more preferably, about 32% v/v of saturated ammonium sulphate.

In preferred embodiments that relate to capsomere preparation, the concentration of ammonium sulphate is at least about 12% v/v of saturated ammonium sulphate. More preferably, the concentration of ammonium sulphate is between about 12% v/v of saturated ammonium sulphate and about 50% v/v of saturated ammonium sulphate. Even more preferably, the concentration of ammonium sulphate is between about 12% v/v of saturated ammonium sulphate and 30% v/v of saturated ammonium sulphate. Yet even more preferably, the concentration is between about 15% v/v and about 25% v/v of saturated ammonium sulphate. In particularly preferred embodiments, the final concentration of ammonium sulphate is about 20% v/v of saturated ammonium sulphate.

In preferred embodiments which relate to ammonium sulphate, the ammonium sulphate may present at a final concentration of about 20% v/v, about 20.5% v/v, about 21% v/v, about 21.5% v/v, about 22% v/v, about 22.5% v/v, about 23% v/v, about 23.5% v/v, about 24% v/v, about 24.2% v/v, about 24.4% v/v, about 24.6% v/v, about 24.8% v/v, about 25% v/v, about 25.2% v/v, about 25.4% v/v, about 25.6% v/v, about 25.8% v/v, about 26% v/v, about 26.2% v/v, about 26.4% v/v, about 26.6% v/v, about 26.8% v/v, about 27% v/v, about 27.1% v/v, about 27.2% v/v, about 27.3% v/v, about 27.4% v/v, about 27.5% v/v, about 27.6% v/v, about 27.7% v/v, about 27.8% v/v, about 27.9% v/v, about 28% v/v, about 28.1% v/v, about 28.2% v/v, about 28.3% v/v, about 28.4% v/v, about 28.5% v/v, about 28.6% v/v, about 28.7% v/v, about 28.8% v/v, about 28.9% v/v, about 29% v/v, about 29.1% v/v, about 29.2% v/v, about 29.3% v/v, about 29.4% v/v, about 29.5% v/v, about 29.6% v/v, about 29.7% v/v, about 29.8% v/v, about 29.9% v/v, about 30% v/v, about 30.1% v/v, about 30.2% v/v, about 30.3% v/v, about 30.4% v/v, about 30.5% v/v, 30.6% v/v, about 30.7% v/v, about 30.8% v/v, about 30.9% v/v, about 31% v/v, about 31.2% v/v, about 31.3% v/v, about 31.4% v/v, about 31.5% v/v, about 31.6% v/v, about 31.7% v/v, about 31.8% v/v, about 31.9% v/v, about 32% v/v, about 32.1% v/v, about 32.2% v/v, about 32.3% v/v, about 32.4% v/v, about 32.5% v/v, about 32.6% v/v, about 32.7% v/v, about 32.8% v/v, about 32.9% v/v, about 33% v/v, about 33.1% v/v, about 33.2% v/v, about 33.3% v/v, about 33.4% v/v, about 33.5% v/v, about 33.6% v/v, about 33.7% v/v, about 33.8% v/v, about 33.9% v/v, about 34% v/v, about 34.1% v/v, about 34.2% v/v, about 34.3% v/v, about 34.4% v/v, about 34.5% v/v, about 34.6% v/v, about 34.7% v/v, about 34.8% v/v, about 34.9% v/v, about 35% v/v, about 35.1% v/v, about 35.2% v/v, about 35.3% v/v, about 35.4% v/v, about 35.5% v/v, about 35.6% v/v, about 35.7% v/v, about 35.8% v/v, about 35.9% v/v, about 36% v/v, about 36.1% v/v, about 36.2% v/v, about 36.3% v/v, about 36.4% v/v, about 36.5% v/v, about 36.6% v/v, about 36.7% v/v, about 36.8% v/v, about 36.9% v/v, about 37% v/v, about 37.1% v/v, about 37.2% v/v, about 37.3% v/v, about 37.4% v/v, about 37.5% v/v, about 37.6% v/v, about 37.7% v/v, about 37.8% v/v, about 37.9% v/v, about 38% v/v, about 38.2% v/v, about 38.4% v/v, about 38.6% v/v, about 38.8% v/v, about 40% v/v, about 41% v/v, about 42% v/v, about 43%, about 44% v/v, about 45%, about 46% v/v, about 47% v/v, about 48% v/v, about 49% and about 50% v/v of saturates ammonium sulphate.

In general aspects, the final concentration of any agent used in the present invention and in particular salt and in preferably ammonium sulphate needed to preferentially retain the virus particle or VLP in the aqueous phase may be different depending on the nature of the particle and/or surface properties of the particle.

In particular embodiments that relate to murine polyomavirus VLPs which further comprise one or more foreign immunogenic epitopes, the ammonium sulphate concentration is preferably between about 20% v/v of saturated ammonium sulphate and 30% v/v of saturated ammonium sulphate, more preferably, between about 25% v/v of saturated ammonium sulphate and 27% v/v of saturated ammonium sulphate, even more preferably about 25% v/v of saturated ammonium sulphate.

In those embodiments that relate to a murine polyomavirus VLP without a foreign epitope, the ammonium sulphate final concentration is between about 28% v/v of saturated ammonium sulphate and 40% v/v of saturated ammonium sulphate, more preferably, between about 24% v/v and about 33% v/v and even more preferably, the ammonium sulphate final concentration is about 32% of saturated ammonium sulphate.

In embodiments that relate to use of ammonium sulphate in capsomere purification, the ammonium sulphate final concentration is between about 12% v/v and about 50% v/v of saturated ammonium sulphate. Even more preferably, the ammonium sulphate concentration is between about 12% and about 30%.

In general embodiments, ‘% v/v’ can relate to the percentage volume of a saturated solution of an agent and in particular a saturated salt solution. In particularly preferred embodiments which relate to ammonium sulphate, ‘% v/v’ can relate to the percentage volume of a saturated ammonium sulphate solution. In particular, a saturated solution of ammonium sulphate (ca. 4M at 4° C.) may be added to a mixture to obtain a final concentration expressed as % v/v of saturated ammonium sulphate.

It will be appreciated that the treatment with an appropriate agent according to the present invention results in an isolated virus particle and/or a VLP being present in solution.

In another broad aspect of the present invention, the invention relates to methods of preparing a capsomere using ion-exchange chromatographic materials.

The present invention further provides chromatographic materials comprising an ion exchanger. The ion exchanger may be a cation exchanger wherein the cation exchanger comprises sulfate, phosphate and carboxylate derivitized chromatographic materials. The ion exchanger may also be an anion exchanger, wherein the anion exchanger comprises positively charged chromatographic material. The positively charged chromatographic material may be quaternary amine (Q) or diethylaminoethane (DEAE).

More specifically, and as applied to the present invention, the basic principle of ion-exchange chromatography is that the affinity of a capsomere for the exchanger depends on both the electrical properties of the protein, and the relative affinity of other charged substances in the solvent. Hence, bound proteins can be eluted by changing the pH, thus altering the charge of the protein, or by adding competing materials, of which salts are but one example. Because different substances have different electrical properties, the conditions for release vary with each bound molecular species. In general, to get good separation, the methods of choice are either continuous ionic strength gradient elution or stepwise elution. For an anion exchanger, either pH is decreased and ionic strength is increased or ionic strength alone is increased. For a cation exchanger, both pH and ionic strength can be increased. The actual choice of the elution procedure is usually a result of trial and error and of considerations of stability of the capsomere being purified. It will be appreciated by a skilled practitioner of this art, that the type of anion-exchanger, and the buffers, and salts used to bind and elute the capsomere will also be a function of the type of capsomere sought to be purified.

It is well known that the principle of ion-exchange chromatography is that charged molecules adsorb to ion exchangers reversibly so that molecules can be bound or eluted by changing the ionic environment. Separation on ion exchangers is usually accomplished in two stages: first, the substance to be separated is bound to the exchanger, using conditions that give stable and tight binding; then the substance is eluted with buffers of different pH, or ionic strength, depending on the properties of the substance being purified.

The anion and cation exchange chromatographic materials can be used in gravity column chromatography or high pressure liquid chromatography apparatus using radial or axial flow, fluidized bed columns, or in a slurry, that is, batch, method. In the latter method, the resin is separated from the sample by decanting or centrifugation or filtration or a combination of methods. The invention also contemplates use of ion exchange membranes and use of ion-exchange monolith technology such as monolith ion exchange columns (eg from BIA Separations) as are well known in the art.

It will be appreciated that anion exchange chromatography uses a positively-charged organic moiety covalently cross-linked to an inert polymeric backbone. The latter is used as a support for the resin. Representative organic moieties are drawn from primary, secondary, tertiary and quaternary amino groups; such as trimethylaminoethyl (TMAE), diethylaminopropyl, diethylaminoethyl (DEAE), dimethylaminoethyl (DMAE), and other groups such as the polyethyleneimine (PEI) that already have, or will have, a formal positive charge within the pH range of approximately 5 to approximately 9. In one embodiment, an anion exchange resin consisting of DMAE, TMAE, DEAE, or quaternary ammonium groups is used. A number of anion exchange resins sold under the tradename Fractogel (Novagen) use TMAE, DEAE, DMAE as the positively-charged moiety, and a methacrylate co-polymer background. Resins that use quaternary ammonium resins and quaternary ammonium resins of the type sold under the trade name Q SOURCE-30 (Amersham Biosciences) may also be employed. Q SOURCE-30 has a support made of polystyrene cross-linked with divinylbenzene.

Several possible anion exchange media are known that can be used in such columns including N-charged amino or imino resins such as POROS 50 PI™, Q SEPHAROSE™, any DEAE, TMAE, tertiary or quaternary amine, or PEI-based resin. One skilled in the art will appreciate that capsomeres can be purified on an anion exchange material either before or after purification on other chromatographic materials or with other purifying agents such as ammonium sulphate.

In cation exchange chromatography, a negative functional group is bound to the insoluble support medium. Accordingly, cation exchange chromatographic media bind positive counter ions when the incubation period is a sufficient time period to allow for the positively charged groups to bind to and come to equilibrium with the negatively charged cation exchanger medium. Neutral molecules and anions do not bind to the cation exchange medium. Following the electrostatic binding of species possessing a net positive charge, the cationic medium is washed, removing non-binding molecules from the medium. Bound ions are then eluted either by washing the medium with increasing concentrations of positive ions or by altering the pH of the medium. The disclosed invention contemplates using a variety of cation exchange media such as any sulfo-, phosphor carboxy-, or carboxy-methyl-based cation exchange resins bound to numerous support medium well known in the art. In one embodiment, the cation exchange chromatographic material consists of sulfopropyl or carboxymethyl. Resins that use cation exchange resins sold under the trade name SP Sepharose™ (Amersham; sulfopropyl) and CM Sepharose™ (Amersham; carboxymethyl) may also be used

It is envisaged that the methods of the invention can be used in combination with any one or more of a number of protein purification techniques or alternatively, the steps of the methods of the invention may be carried out alone. In one embodiment of the invention, one or more steps preceding the step of contacting a mixture with an agent as herein described may be desirable to reduce the load challenge of the contaminants or impurities. In another embodiment of the invention, one or more purification steps following the step of contacting a mixture with an agent as herein described may be desirable to remove additional contaminants or impurities, or to further concentrate the isolated virus particle and/or the VLP preparation.

In broad aspects that relate to method of a preparing a capsomere that is substantially-free of one or more host cell derived chaperone proteins, the method including the step of contacting a mixture comprising a capsomere and at least one host cell derived chaperone protein with an agent selected from the group consisting of an anion exchanger chromatographic material, a cation exchanger chromatographic material, ammonium sulphate, a PEG and combinations thereof, such that the capsomere is selectively separated from at least one host cell derived chaperone protein.

It will be appreciated that in the methods of the invention, use of these agents may be alone or combination with each other, or may occur in an ordered sequence such as a step-wise fashion although not limited thereto.

In preferred embodiments of the present invention which relate to methods preparing a capsomere that is substantially-free of one or more host cell derived proteins which includes the steps of subjecting a mixture comprising a capsomere and one or more host cell derived chaperone protein to any one or a plurality of the following treatments:
(a) contacting said mixture with an anion ion-exchange chromatographic material to selectively separate the capsomere from at least one host cell derived chaperone protein;
(b) contacting said mixture with a cation ion-exchange chromatographic material to selectively separate the capsomere from at least one host cell derived chaperone protein; and
(c) contacting said mixture with ammonium sulphate or PEG, preferably ammonium sulphate, to selectively separate the capsomere from at least one host cell derived chaperone protein.

In preferred forms of these embodiments, the method comprises (a); and (b) or (c). In particularly preferred forms, the sequence of step is (a) followed by (b) or (c).

In other preferred forms, the method comprises (c) followed by (a) or (b).

In preferred embodiments, step (c) uses ammonium sulphate.

The method of the present invention may optionally be combined with other purification steps, including but not limited to, Protein A chromatography, affinity chromatography, hydroxyapatite chromatography, hydrophobic interaction chromatography, immobilized metal affinity chromatography, size exclusion chromatography, diafiltration, ultrafiltration, viral removal filtration, and/or ion exchange chromatography. Further purification methods may include filtration, precipitation, evaporation, distillation, drying, gas absorption, solvent extraction, press extraction, adsorption, crystallization, and centrifugation. Other purification methods may include further chromatography according to this invention utilizing batch or column chromatography. In addition, further purification can include combinations of any of the foregoing, such as for example, combinations of different methods of chromatography, combinations of chromatography with filtration, combinations of chromatography with precipitation, or combinations of membrane treatment with drying.

The quality or integrity of molecules prepared according to the methods of the present invention can be monitored by techniques known in the art including optical density, transmission electron microscopy, or light scattering. Additionally, the biological properties of the particles prior to and after treatment can be determined using well established assays. More specifically, the purity and identity may be measured using a variety of analytical methods including, reduced and non-reducing sodium dodecyl sulfate polyacrylamide gel electrophoresis (SDS-PAGE), size exclusion chromatography, HPLC (high performance liquid chromatography), capillary electrophoresis, MALDI (Matrix Assisted Laser Desorption Ionization) mass spectrometry, ELISA (Enzyme Linked Immunosorbent Assay), Asymmetric-Flow Field-Flow-Fractionation (AF4) or circular dichroism. In particularly preferred embodiments, AF4 is used to monitor the capsomeres, virus particles or VLPs as well as misformed and/or aggregated particles or VLPs.

The methods of the present invention are suitably applicable to any isolated capsomere, virus particle or a VLP. In preferred embodiments, the capsomere, virus particle or VLP comprises one or more isolated proteins comprising a polyomavirus VP1 amino acid sequence and more preferably, comprising a murine polyomavirus VP1 amino acid sequence. According to other general preferred embodiments, the capsomere, virus particle or VLP comprises one or more isolated proteins comprising a human papillomavirus (HPV) capsid protein amino acid sequence. More preferably, the HPV capsid protein amino acid sequence is selected from L1 and L2. Even more preferably, the HPV capsid protein amino acid sequence is L1.

The invention also contemplates capsomeres, virus particles or VLPs derived from derivatives of one or a plurality of virus structural proteins or comprising one or more derivatives. As used herein, “derivative” proteins of the invention have been altered, for example by addition, conjugation or complexing with other chemical moieties or by post-translational modification techniques as are well understood in the art.

It will further be appreciated that the particle structures of the invention may comprise one or more additional amino acid sequences other than the virus structural proteins. “Additions” of amino acids may include fusion of a virus structural protein of the invention or a fragment thereof with other proteins or peptides. The other protein may, by way of example, assist in the purification of the protein. For instance, these include a polyhistidine tag, maltose binding protein (MBP), green fluorescent protein (GFP), Protein A or glutathione S-transferase (GST). Other additions include “epitope tags” such as FLAG and c-myc epitope tags.

Well known examples of fusion partners include, but are not limited to, glutathione-S-transferase (GST), Fc portion of human IgG, maltose binding protein (MBP) and hexahistidine (HIS₆), which are particularly useful for isolation of the fusion protein by affinity chromatography. For the purposes of fusion protein purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system.

In some cases, the fusion partners also have protease cleavage sites, such as for Factor Xₐ or Thrombin, which allow the relevant protease to partially digest the fusion protein of the invention and thereby liberate the recombinant protein of the invention therefrom. The liberated protein can then be isolated from the fusion partner by subsequent chromatographic separation.

Alternatively, the additional proteins or peptides include immunogenic or antigenic epitopes. Said epitopes are generally included to induce a corresponding immune response including a humoral and/or T-cell mediated immune response. The desired immune response may be directed against one or more pathogens, although without limitation thereto. Therefore it is contemplated that the epitopes may be endogenous to a VLP or alternatively, may be directed to or derived from foreign pathogens (which may be referred to as a ‘chimera’). The invention contemplates immunogenic epitopes derived from foreign pathogens inclusive of viruses and bacteria. In general preferred embodiments, the immunogenic epitopes are derived from influenza virus. Reference is made to International Publication No. WO2008/040060 which provides non-limiting examples of suitable influenza immunogenic epitopes and is incorporated herein by reference. In other general embodiments, the immunogenic epitopes are derived from Hendra virus and/or Group A Streptococcus pyogenes.

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention. An example of methods suitable for chemical derivatization of proteins is provided in Chapter 15 of CURRENT PROTOCOLS IN PROTEIN SCIENCE Eds. Coligan et. al., John Wiley & Sons NY (1995-2008).

The virus particles, capsomeres and/or VLPs prepared by the methods of the present invention can be utilised in a number of applications but are particularly useful in pharmaceutical compositions and in methods of therapy and/or prophylaxis.

The composition may be used in therapeutic or prophylactic treatments as required.

A preferred form of a pharmaceutical composition is an immunotherapeutic composition. An immunotherapeutic composition preferably is a vaccine.

Suitably, the pharmaceutical composition comprises a pharmaceutically-acceptable carrier. By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water.

Any suitable route of administration may be employed for providing a patient with the pharmaceutical composition of the invention. For example, intranasal, transdermal, oral, rectal, parenteral, sublingual, buccal, intravenous, intra-articular, intra-muscular, intra-dermal, subcutaneous, inhalational, intraocular, intraperitoneal, intracerebroventricular and the like may be employed. Intranasal, transdermal, intra-muscular and subcutaneous application may be appropriate for administration of immunogenic agents of the present invention. Intranasal and transcutaneous administration in one preferred form includes use of cholera toxin and CpG-oligonucleotides as adjuvants. Another particularly preferred form of the present invention is intranasal administration of unadjuvanted particles (such as compositions comprising particles in PBS) and preferably, VLPs. CpG-oligonucleotides are thought to induce primarily a Th1 immune response and cholera toxin is thought to induce mucosal IgA when administered orally or intranasally, but induces an IgG response when administered transcutaneously, see for example Berry et al, 2004, Infect Immun 72 1019, incorporated herein by reference. Skin penetration enhancers, such as chemical penetration enhancers including DMSO and electrically assisted methods including iontohoresis, may also be used as described for example in Barry, 2004, Nature Biology 22 165.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be affected by using other polymer matrices, liposomes and/or microspheres.

Pharmaceutical compositions of the present invention suitable for administration may be presented as discrete units such as vials, capsules, sachets or tablets each containing a pre-determined amount of one or more immunogenic agent of the invention, as a powder, or granules, or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more immunogenic agents as described above with the carrier, which constitutes one or more necessary ingredients. In general, the compositions are prepared by uniformly and intimately admixing the agents of the invention with liquid carriers or finely divided solid carriers or both, and then, if necessary, shaping the product into the desired presentation.

The present invention also contemplates methods of treatment, method of eliciting an immune response and/or methods of immunising an animal which include the step of administering a pharmaceutical composition as hereinbefore described.

An immune response includes within its scope a humoral and/or T-cell mediated immune response.

It will be appreciated that the methods of the present invention may be performed in a batch format or alternatively as a continuous system.

So that the invention may be readily understood and put into practical effect, the following non-limiting Examples are provided.

### EXAMPLES

### Example 1

Recombinant VP1 protein was expressed as a GST fusion in Escherichia coli (Chuan et al., 2008a) and purified to yield pentameric VP1 protein capsomeres (Lipin et al., 2008). Capsomeres collected following size-exclusion chromatography on a Superdex 200 column (GE Healthcare Biosciences, Buckinghamshire, UK) were used to assemble VLPs by dialysis against GL1 buffer (20 mM Tris pH 7.4, 5% v/v glycerol, 1 mM CaCl₂, 500 mM (NH₄)₂SO₄) for 15 h and then against GL2 buffer (20 mM Tris pH 7.4, 200 mM NaCl, 5% glycerol, 1 mM CaCl₂) for 24 h. Analysis of capsids with AF4 was as previously described (Chuan et al., 2008b). Hydrodynamic radius of capsids following AF4 fractionation was measured with dynamic light-scattering using a Wyatt-QELS system (Wyatt Technology Corporation). GL2 buffer was used for AF4 analysis.

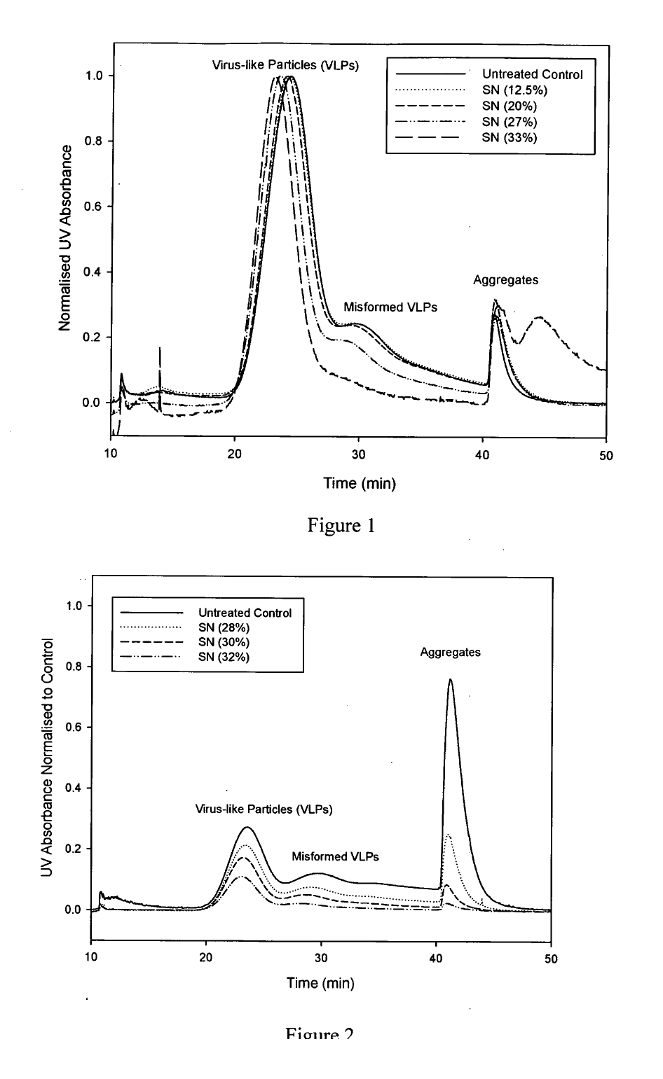

Treatment of VLPs with (NH₄)₂SO₄ at 12.5%, 20%, 27%, 33% or 40%. (FIG. 1)

VLP sample (1.1 mL) diluted to 500 μg mL⁻¹ with GL2 buffer was centrifuged (15000 rpm, 4° C., 5 min). Supernatant comprising 5 aliquots of 200 and 1 aliquot of 80 μL (“Untreated Control”) was recovered. Saturated (NH₄)₂SO₄ solution was added to the 200 μL aliquots to obtain a final concentration of 12.5%, 20%, 27%, 33% or 40% (all % v/v) of saturated (NH₄)₂SO₄. Mixtures were incubated on a roller mixer (4° C., 1 h) and then centrifuged (10000 rpm, 4° C., 10 min). Supernatant was collected and the precipitate was re-suspended in 100 μL GL2 buffer. Samples were centrifuged (15000 rpm, 4° C., 5 min) and each supernatant was analysed by AF4.

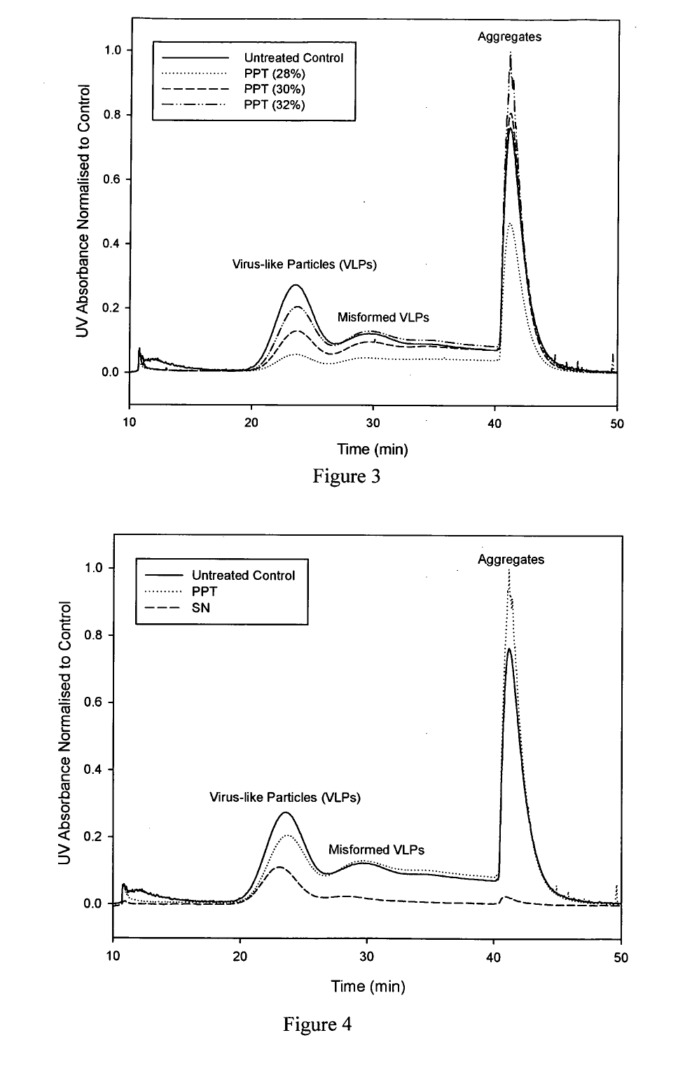

Treatment of VLPs with (NH₄)₂SO₄ at 28%, 30% or 32%. (FIGS. 2-4)

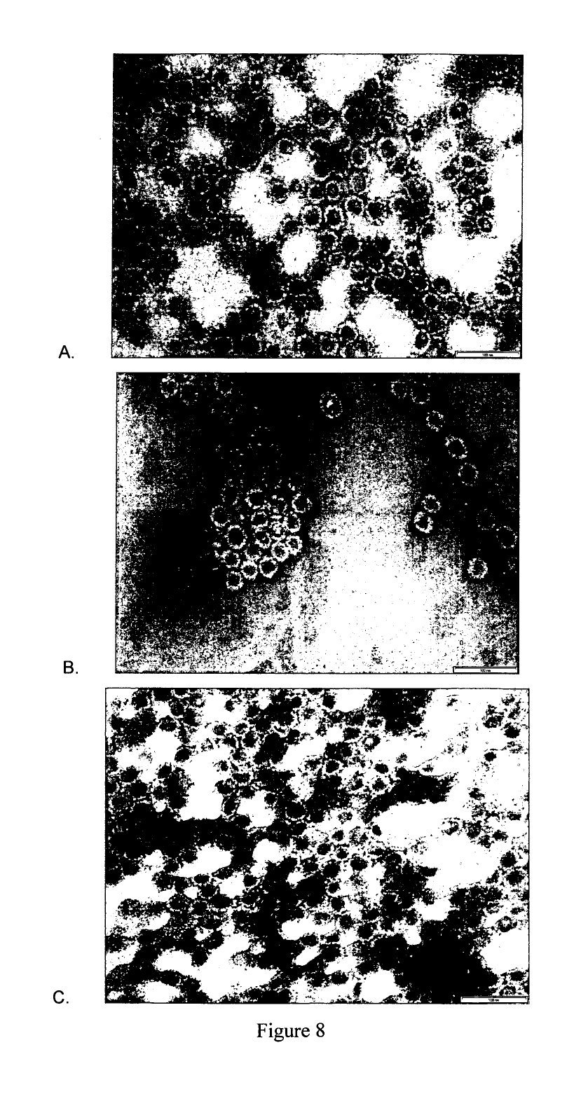

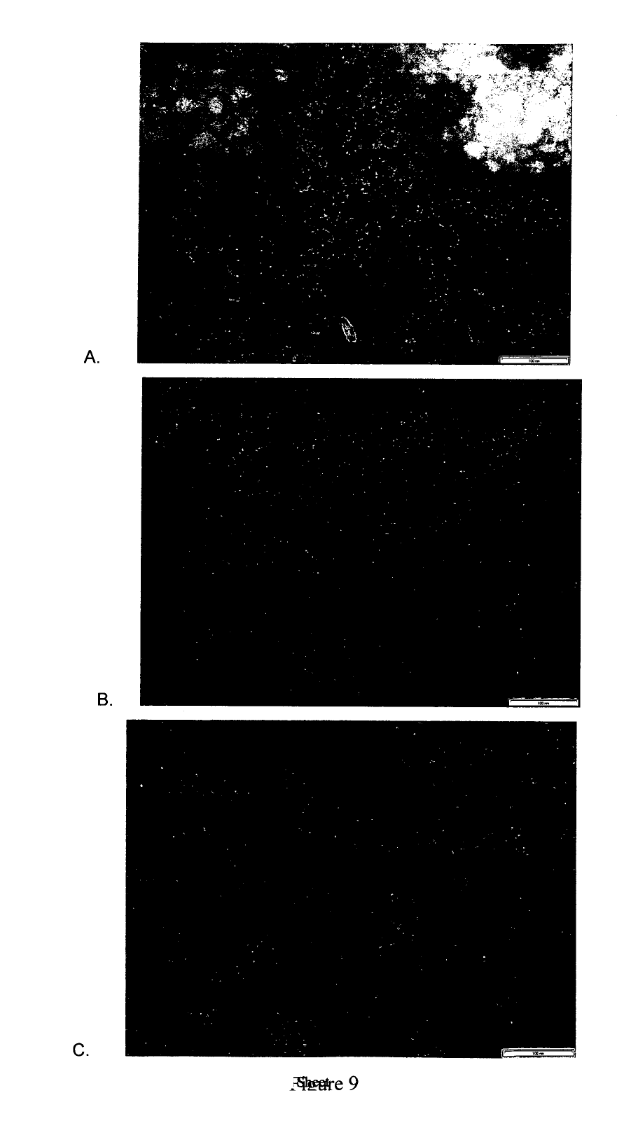

VLP sample (0.72 mL) diluted to 600 μg mL⁻¹ with GL2 buffer was centrifuged (15000 rpm, 4° C., 5 min). Supernatant comprising 3 aliquots of 200 μL and 1 aliquot of 80 μL (“Untreated Control”) was recovered. Saturated (NH₄)₂SO₄ solution was added to the 200 μL aliquots to obtain a final concentration of 28%, 30% or 32% (all % v/v) of saturated (NH₄)₂SO₄. Mixtures were incubated on a roller mixer (4° C., 1 h) and then centrifuged (10000 rpm, 4° C., 10 min). Supernatant was collected and the precipitate was re-suspended in 100 μL GL2 buffer. Samples were centrifuged (15000 rpm, 4° C., 5 min) and each supernatant was then analysed by AF4. The results for each concentration are shown in FIGS. 2 to 4 respectively. FIG. 8 are transmission electron micrographs of the SN and PPT resulting from 32% (NH₄)₂SO₄ which visually confirming the quality metrics of the preparations.

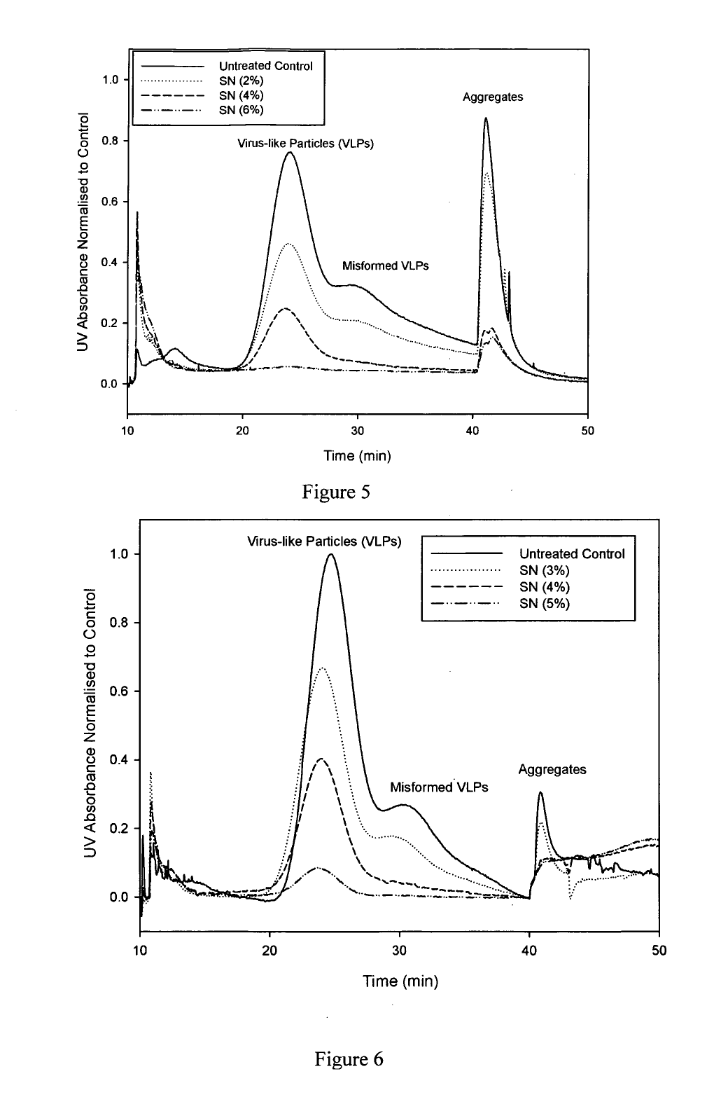

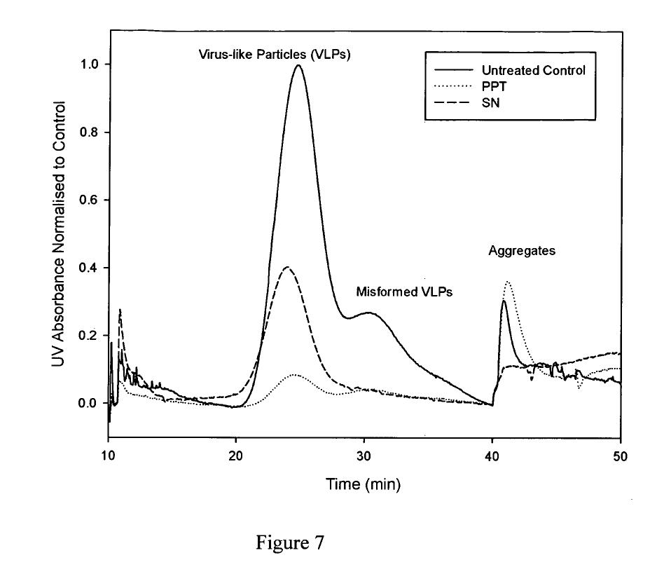

Treatment of VLPs with poly-ethylene glycol (PEG) at 2%, 4% or 6%. (FIG. 5)

VLP sample (1.0 mL) diluted to 750 μg mL⁻¹ with GL2 buffer was centrifuged (15000 rpm, 4° C., 5 min). Supernatant comprising 3 aliquots of 200 and 1 aliquot of 100 μL (“Untreated Control”) was recovered. PEG 6000 powder (Fluka AR 81260) was carefully weighed before directly adding the 200 μL aliquots to obtain a final concentration of 2%, 4% or 6% (all % w/v) of PEG. Mixtures were incubated on a roller mixer (4° C., 1.5 h) and then centrifuged (10000 rpm, 4° C., 10 min). Supernatant was collected and the precipitate was re-suspended in 200 μL GL2 buffer. Samples were centrifuged (15000 rpm, 4° C., 5 min) and each supernatant was then analysed by AF4.

Treatment of VLPs with poly-ethylene glycol (PEG) at 3%, 4% or 5%. (FIGS. 6 & 7)

VLP sample (1.1 mL) diluted to 545 μg mL⁻¹ with GL2 buffer was centrifuged (15000 rpm, 4° C., 5 min). Treatment was as per Example 3, except that 3%, 4% or 5% (all % w/v) of PEG was used. FIG. 9 are transmission electron micrographs of the SN and PPT resulting from 4% PEG treatment which visually confirming the quality metrics of the preparations.

### Discussion

The results shown herein demonstrate that the VLPs elute before misformed VLPs and then those elute before aggregates. We see enrichment of VLPs in the supernatant and enrichment of aggregates and misformed VLPs into the precipitate. The first figure is height normalised to emphasise the shift in distribution—obviously the peak height if not normalised decreases, as in the other figures (because material partitions from the feed into either the supernatant or precipitate phases).

### Example 2

A mixture comprising virions will be treated an agent in order to selectively separate virions from a heterogenous population.

### Example 3

Treatment of Chimeric VLPs with (NH₄)₂SO₄ at 23%, 25%, 27%, 29% or 31%.

Wild type VP1 carrying influenza antigen (herein referred to as “chimeric” VLPs, sequence designation H5AQG4S) were treated with various concentration of (NH₄)₂SO₄. H5AQG4S amino acid sequence is as follows (underlined is the amino acid sequence of avian influenza HA antigen): GGGGSPYQGKSSGGGGS (SEQ ID NO: 1)

Chimeric VLP (H5AQG4S) sample (0.6 mL) at the concentration of 400 μg mL⁻¹ was centrifuged (15000 rpm, 4° C., 5 min). Supernatant comprising 5 aliquots of 100 μL and 1 aliquot of 80 μL (“Untreated Control”) was recovered. Saturated (NH₄)₂SO₄ solution was added to the 100 μL aliquots to obtain a final concentration of 23%, 25%, 27%, 29% or 31% (all % v/v) of saturated (NH₄)₂SO₄. Mixtures were incubated on a roller mixer (4° C., 1 h) and then centrifuged (10000 rpm, 4° C., 10 min).

Supernatant was collected and the precipitate was re-suspended in 100 μL GL2 buffer. Samples were centrifuged (15000 rpm, 4° C., 5 min) and each supernatant was then analysed by EM at 100 000 magnification. High-quality VLPs were recovered following treatment with 25-27% concentration of (NH₄)₂SO₄.

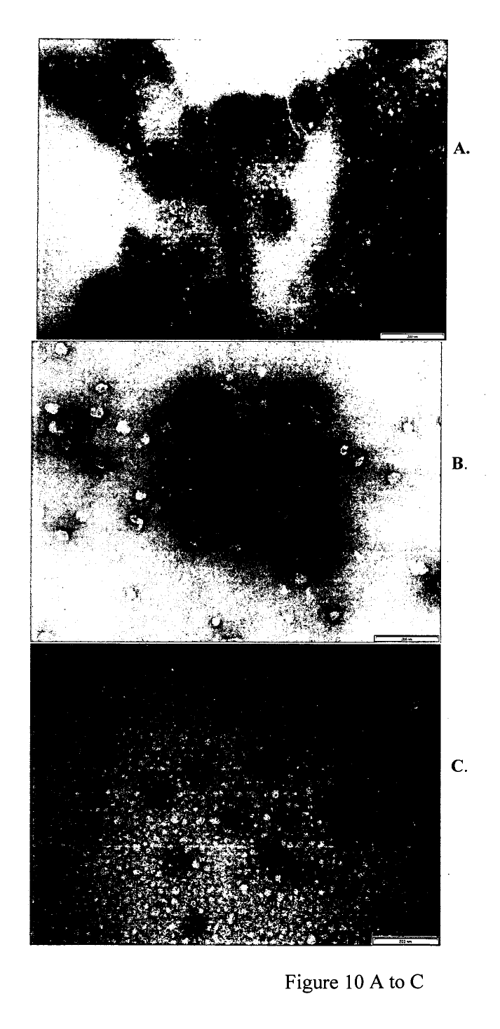

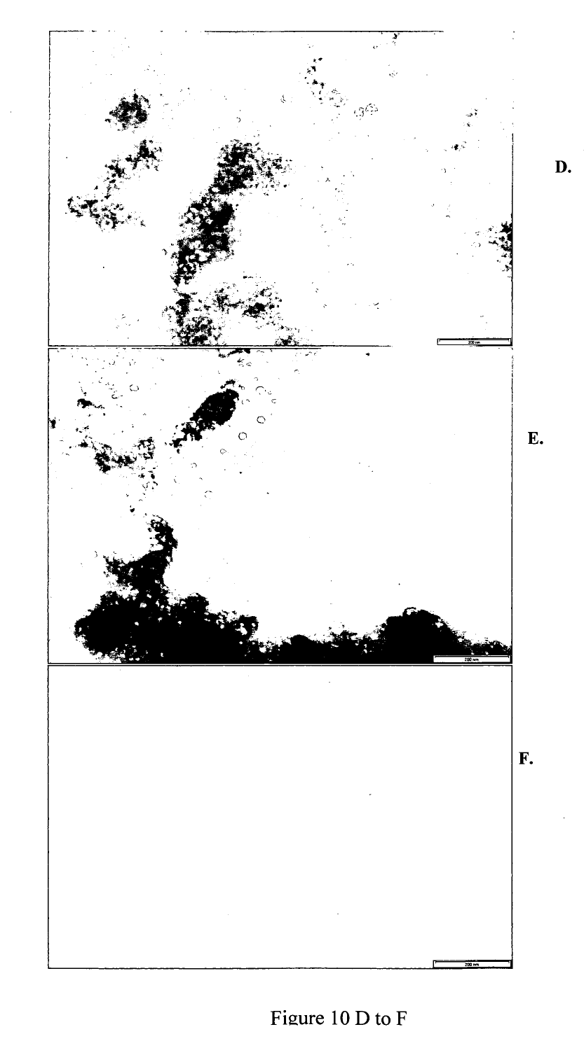

The results are shown in FIG. 10 panels A to F. The control shows a very heterogeneous VLP preparation which is fractionated into correct VLPs by treatment. FIG. 10C shows very homogeneous VLPs in the supernatant (aqueous phase) following treatment with 25% v/v ammonium sulphate. At higher concentrations of ammonium sulphate, VLPs start to self-associate (aggregate) until at 31% v/v no VLPs remain in the supernatant (FIG. 10F).

### Example 4

### Capsomere Purification

### Materials and Methods

Recombinant VP1 protein was expressed as a GST fusion in Escherichia coli (Chuan et al., 2008a) and purified to yield pentameric VP1 protein capsomeres (Lipin et al., 2008). Capsomeres were used to assemble VLPs by dialysis against GL1 buffer (20 mM Tris pH 7.4, 5% v/v glycerol, 1 mM CaCl₂, 500 mM (NH4)2SO4) for 15 h and then against GL2 buffer (20 mM Tris pH 7.4, 200 mM NaCl, 5% glycerol, 1 mM CaCl₂) for 24 h. Assembled VLPs were analysed by both asymmetric field-flow fractionation (AFFF, Wyatt Technologies) and electron microscopy (EM). Analysis of capsids with AF4 was as previously described (Chuan et al., 2008b). Hydrodynamic radius of capsids following AF4 fractionation was measured with dynamic light-scattering using a Wyatt-QELS system (Wyatt Technology Corporation). GL2 buffer was used for AF4 analysis. For EM, 2 μL of samples were applied to glow-discharged, Formvar carbon-coated grids. The remaining liquid on the grids were drained off after 2 mins, and the grids were then negatively stained with 2% uranyl acetate for 20 s. The samples on the grids were viewed under the Philips TECNAI 12 electron microscope and digital images were acquired using the integrated CCD camera and image acquisition software.

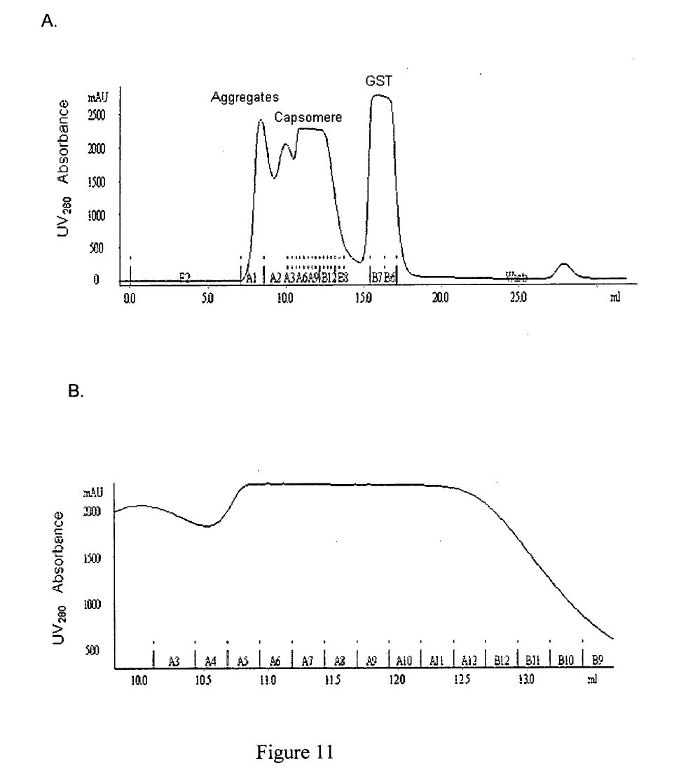

### Identification of Co-purified Contaminants (FIGS. 11 to 13)

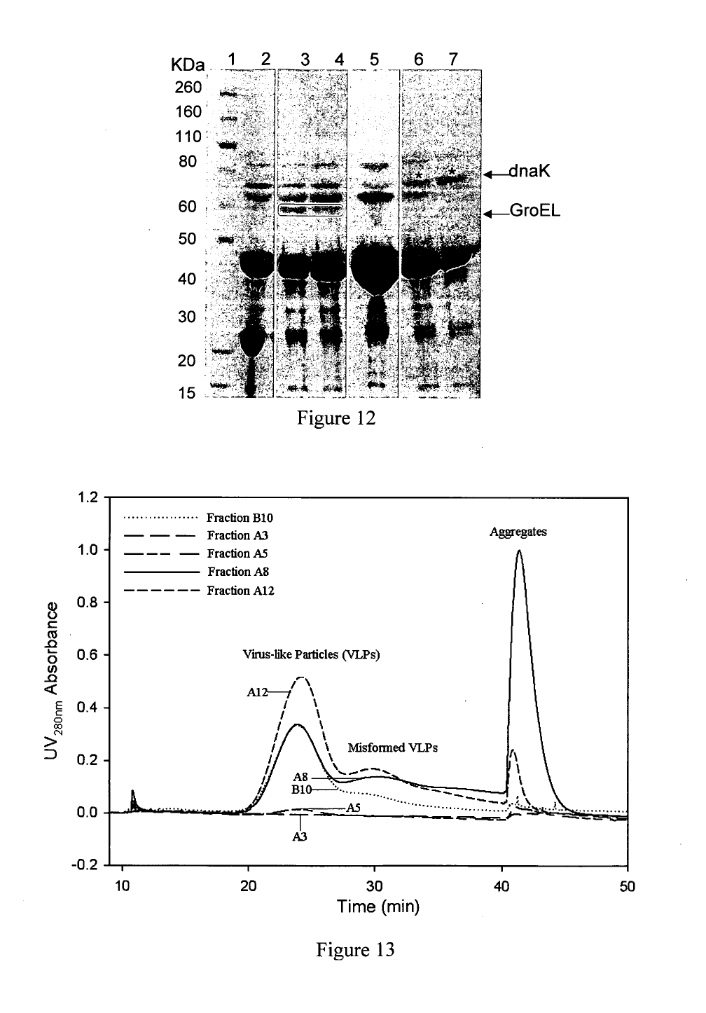

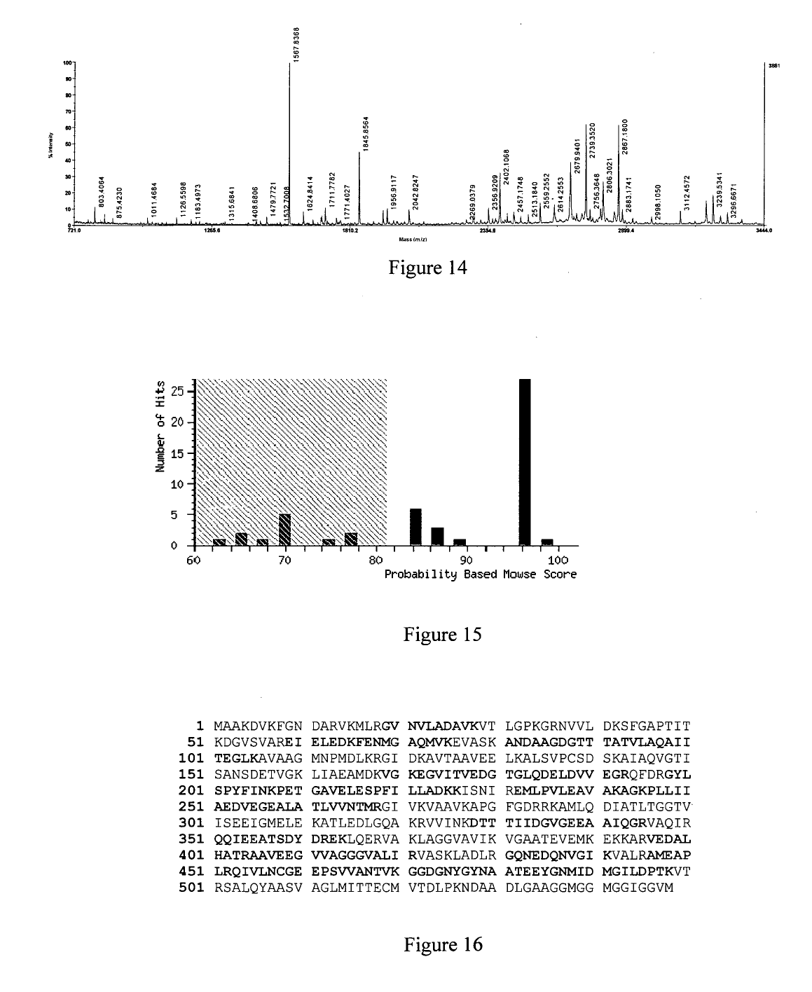

GSTVP1 (960 μL) treated with thrombin was injected onto a Superdex S200 10/300 GL size exclusion column (GE Healthcare) equilibrated in L buffer (40 mM Tris pH 8.0, 200 mM NaCl, 5% glycerol, 1 mM EDTA) to separate capsomeres from aggregates, GST and thrombin (FIG. 11). The purified capsomere fractions were analysed using SDS-PAGE gel electrophoresis (FIG. 12) followed by MALDI-MS (FIGS. 14 to 19; Tables 1 to 4)) to identify contaminants. The bands indicated in FIG. 12 with a box and asterisk were sent for peptide mass fingerprinting. Capsomeres from different fractions were assembled into VLPs by dialysis. Then the VLPs were centrifuged (15000 rpm, 4° C., 5 min) and each supernatant was analysed by AF4 (FIG. 13). The results clearly show a detrimental effect of GroEL on VLP assembly with a possible bad effect also of dnaK (FIG. 13).

It is apparent from AF4 analysis (FIG. 13) that fraction B 10 yields product with less misformed VLPs and aggregates, compared with other fractions. FIG. 12 (lane 7) shows this fraction has reduced GroEL levels. In contrast, fractions A3 and A5 yield no detectable VLP (FIG. 13) and are heavily contaminated with GroEL (FIG. 12).

### Peptide Mass Fingerprinting Results

### GroEL

For bands indicated in FIG. 12 with a box, a clear (statistically confident) match was found to the 60 kDa GroEL chaperonin protein from E. coli. See FIGS. 14, 15 and 16 and Tables 1 and 2. Database used in the analysis was LudwigNR Q308_generic_forward (7101271 sequences; 2477103392 residues). Top Score was 99 for Q6UDB4, tr|Q6UDB4|60 kDa chaperonin (Fragment). [Escherichia coli]. Using Probability Based Mowse Score, a protein score is −10*Log(P), where P is the probability that the observed match is a random event. Protein scores greater than 81 are significant (p<0.05). A significant match to Q6UDB4 Score: 99 (Expect: 0.00096) tr|Q6UDB4|60 kDa chaperonin (Fragment). [Escherichia coli] was found. This protein had the following properties: nominal mass (Mᵣ): 57268; Calculated pI value: 4.88. Based on a NCBI BLAST search of Q6UDB4 against nr Taxonomy: Escherichia coli (Variable modifications: Carbamidomethyl (C), Oxidation (M); Cleavage by Trypsin: cuts C-term side of KR unless next residue is P; Number of mass values searched: 56; Number of mass values matched: 19; Sequence Coverage: 48%). FIG. 16 shows matched peptides in Bold.

dnaK

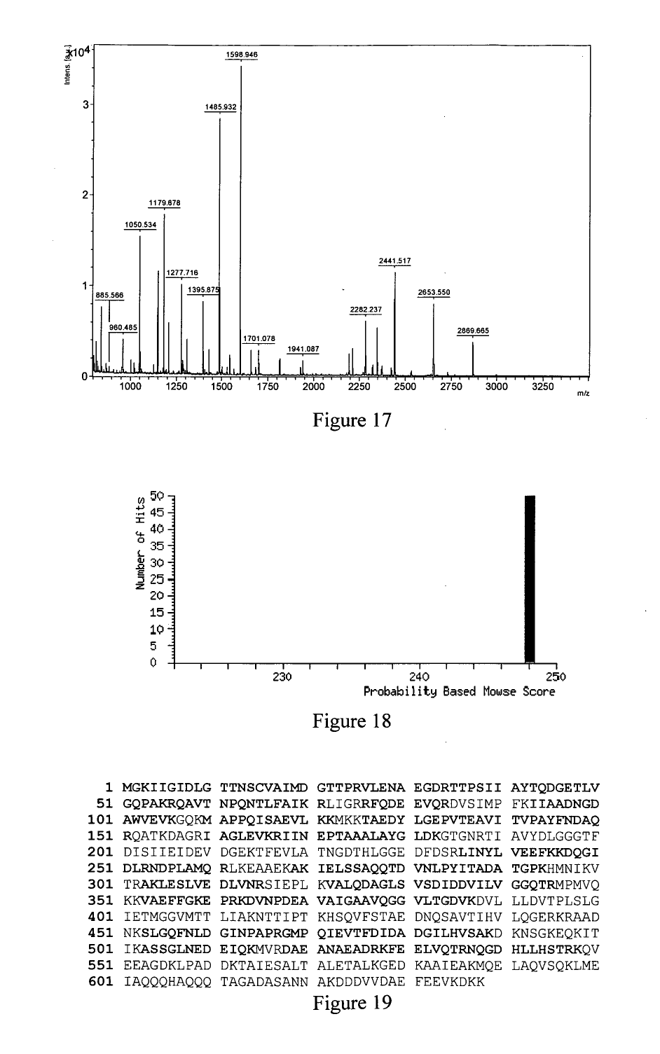

For bands marked with asterisk in FIG. 12, a clear (statistically confident) match was found to the 70 kDa dnaK chaperonin protein from E. coli. See FIGS. 17 to 19 and Tables 3 and 4. The database used for analysis was LudwigNR Q409m_generic_forward (10812415 sequences; 3736504226 residues). The top score was 248 for A7ZHA4, sp|A7ZHA4|Chaperone protein dnaK Tax_Id=331111 [Escherichia coli O139:H28]. Using Probability Based Mowse Score, the Protein score is −10*Log(P), where P is the probability that the observed match is a random event. Protein scores greater than 83 are significant (p<0.05). A match to: A7ZHA4 (Score: 248 Expect: 1.7e-018) sp|A7ZHA4|Chaperone protein dnaK Tax_Id=331111 [Escherichia coli O139:H28] was found with the following characteristics: Nominal mass (Mᵣ): 69130; Calculated pI value: 4.83. An NCBI BLAST search of A7ZHA4 against nr was conducted with the results shown in FIG. 19. The following parameters were used: Fixed modifications: Carbamidomethyl (C); Variable modifications: Oxidation (M); Cleavage by Trypsin: cuts C-term side of KR unless next residue is P; Number of mass values searched: 47; Number of mass values matched: 33; Sequence Coverage: 56%. The matched peptides in FIG. 19 are shown in Bold.

### Example 5

### Capsomere Purification by (NH₄)₂SO₄

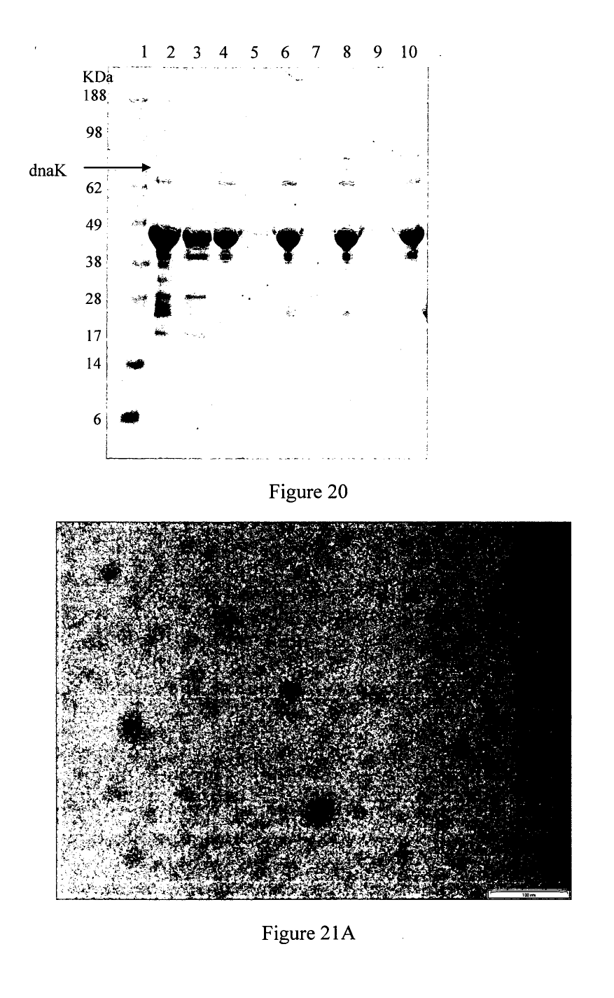

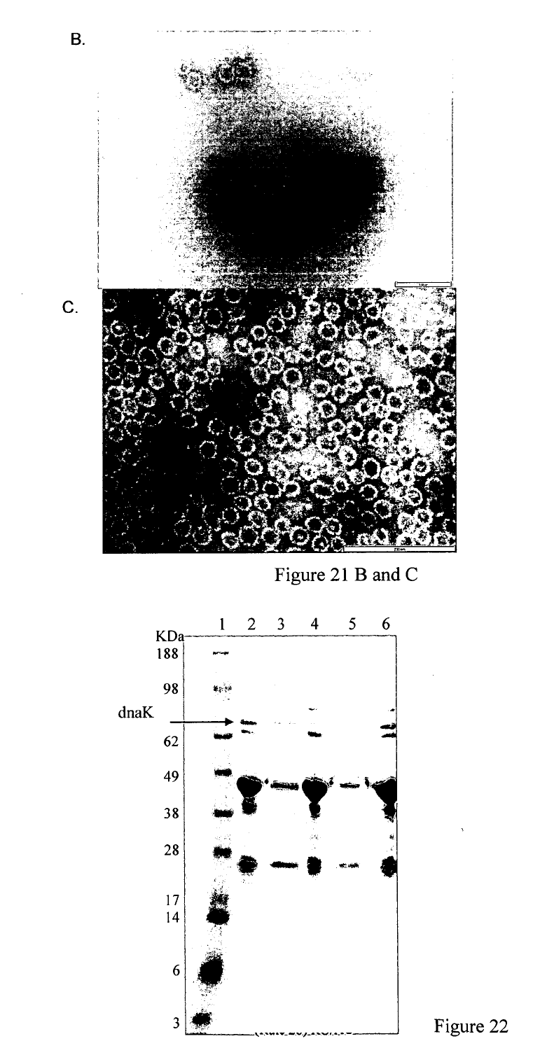

Treatment of VP1 Purified from Size-Exclusion Chromatography with (NH₄)₂SO₄ at 12.5%, 20%, 25% or 30%. (FIGS. 20 and 21)

VP1 sample (0.45 mL) at a concentration of 800 μg mL⁻¹ purified from size-exclusion chromatography (S200VP1) was centrifuged (15000 rpm, 4° C., 5 min). Supernatant comprising 4 aliquots of 100 μL and 1 aliquot of 40 μL (“Untreated Control”) was recovered. Saturated (NH₄)₂SO₄ solution was added to the 100 μL aliquots to obtain a final concentration of 12.5%, 20%, 25% or 30% (all % v/v of saturated (NH₄)₂SO₄). Mixtures were incubated on a roller mixer (4° C., 1 h) and then centrifuged (10000 rpm, 4° C., 10 min). Supernatant was collected and the precipitate was re-suspended in 100 μL L buffer. Samples before and after assembly were analysed by TEM and SDS-PAGE gel electrophoresis.

After treatment with 25% v/v ammonium sulphate, VP1 capsomeres show reduced dnaK levels (FIG. 20, lane 8) and appear relatively homogenous (FIG. 21A). When assembled, these purified capsomeres yield a VLP preparation (FIG. 21B) that is more homogenous than a product assembled from untreated capsomeres (FIG. 21C).

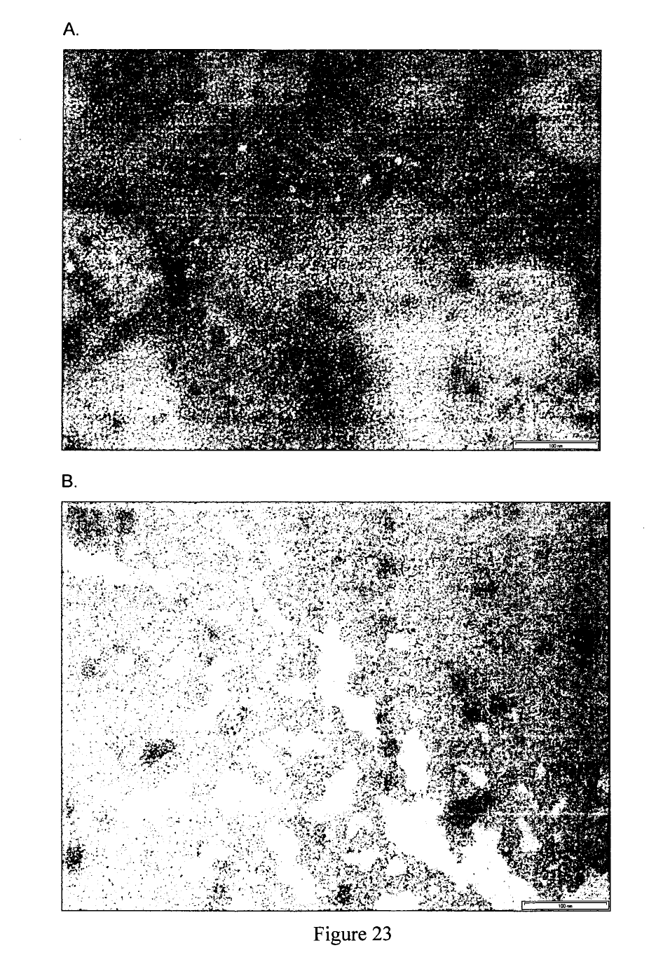

Treatment of VP1 purified from IEX chromatography with (NH₄)₂SO₄ at 25% or 50%. (FIGS. 22 and 23)

VP1 sample (0.45 mL) of the concentration 500 μg mL⁻¹ purified by Q-Sepharose Fast Flow (QFF) ion-exchange chromatography (QFFVP1) (the process is described below) was centrifuged (15000 rpm, 4° C., 5 min). Supernatant comprising 2 aliquots of 200 μL and 1 aliquot of 40 μL (“Untreated Control”) was recovered. (NH₄)₂SO₄ powder was added to the 200 μL aliquots to obtain a final concentration of 25% or 50% (all % v/v) of saturated (NH₄)₂SO₄. Mixtures were incubated on a roller mixer (4° C., 1 h) and then centrifuged (10000 rpm, 4° C., 10 min). Supernatant was collected and the precipitate was re-suspended in 100 μL L buffer. Samples were then analysed by TEM and SDS-PAGE gel electrophoresis. This result confirms the existence of an optimal (NH₄)₂SO₄concentration for dnaK removal.

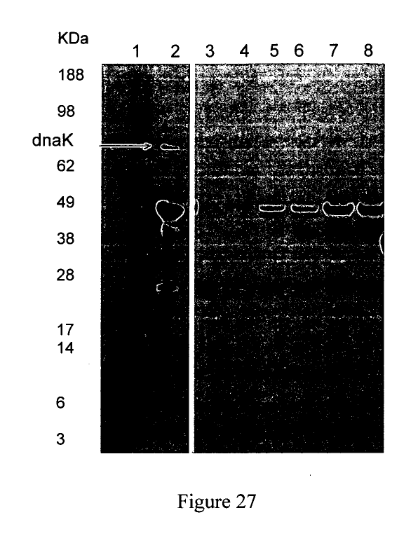

The results show that treatment with 50% v/v ammonium sulphate (FIG. 22, lane 6) precipitates all VP1 protein and contaminants without purification. Conversely, treatment with 25% v/v ammonium sulphate recovers VP1 capsomeres having reduced dnaK levels (FIG. 27, lane 4). The capsomeres after 25% v/v ammonium sulphate treatment exhibit improved uniformity (FIG. 23).

### Example 6

### Capsomere Purification by IEX Chromatography

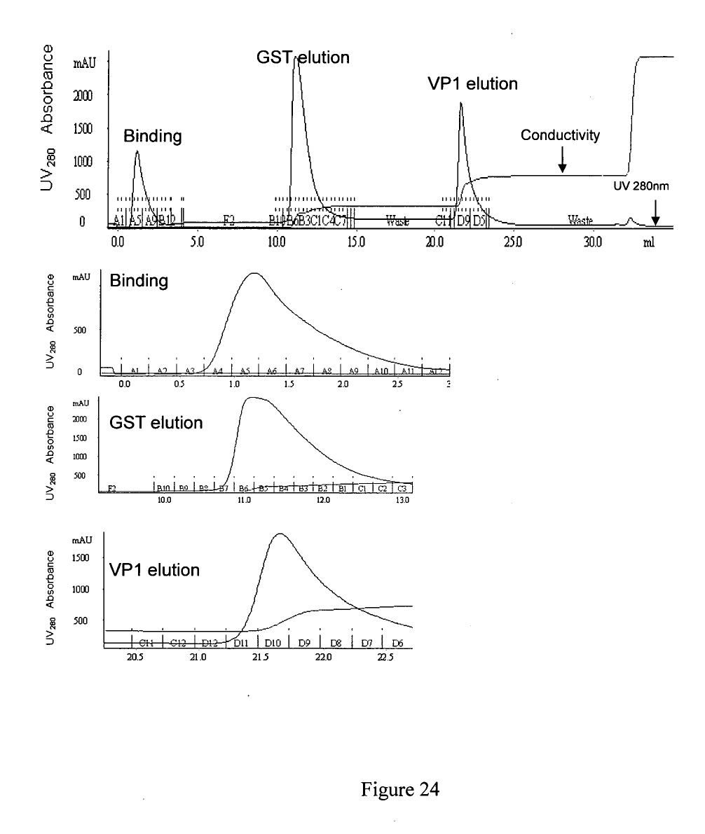

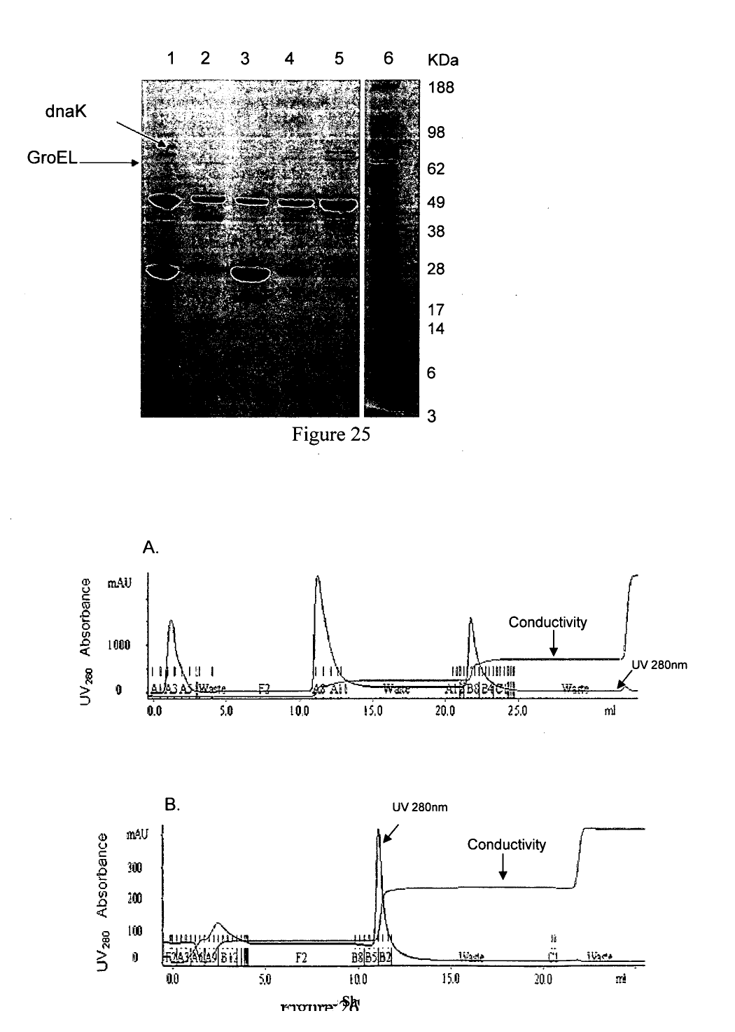

### 1. IEX Chromatography Using QFF Column (FIGS. 24 and 25)

Recombinant VP1 protein was expressed as a GST fusion in Escherichia coli (Chuan et al., 2008a). After purification using GSTrap column and digestion using Thrombin, Capsomeres were separated from GST and aggregates by IEX chromatography using a QFF column. The fractions collected during the binding and elution steps were analysed using SDS-PAGE gel electrophoresis. This result shows GroEL was removed by QFF chromatography.

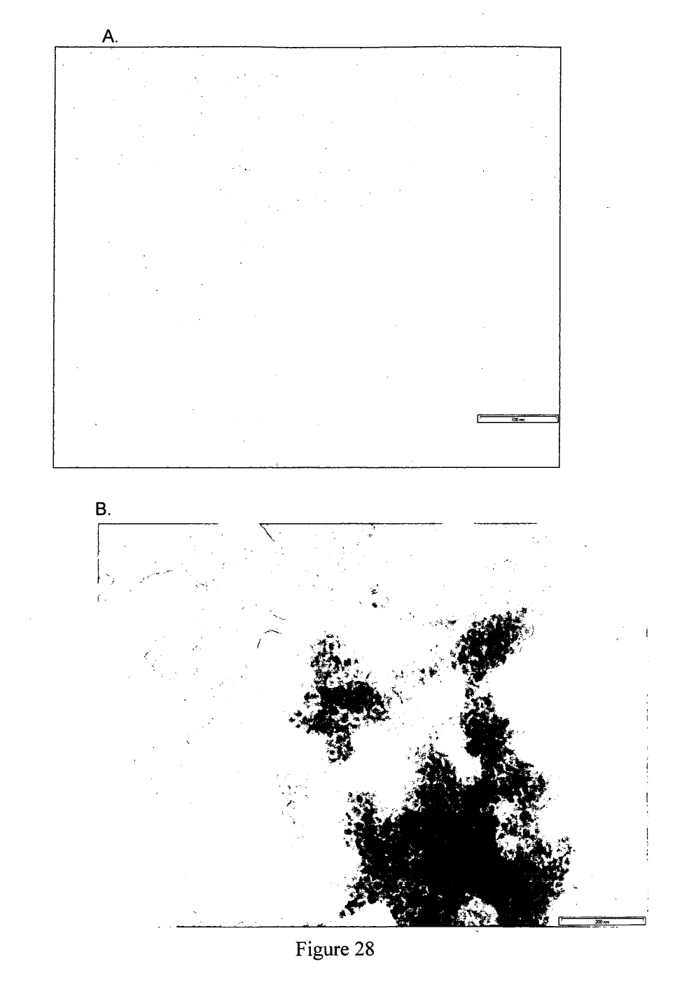

### 2. IEX Chromatography Using SP Column (FIGS. 26 to 28)

QFFVP1 Capsomeres (1.8 mL) were collected from IEX chromatography using QFF column by pooling fractions B8 to B1 (0.25 mL/fraction) altogether. 450 μL of the sample was purified using (NH₄)₂SO₄ (as described above). 1.0 mL of the sample was injected onto an SP column. The fractions collected during the binding, washing and elution steps were analysed using SDS-PAGE electrophoresis. The capsomeres purified from IEX chromatography using QFF and SP column were then assembled into VLPs by dialysis against GL buffer 1 and GL buffer 2 and then analysed by TEM at 100 000 magnification. The result shows that 2-step IEX chromatography yields high-quality capsomeres free of both GroEL and dnaK which yield high-quality VLPs following dialysis into assembly conditions.

### Example 7

### Capsomere Purification by (NH₄)₂SO₄

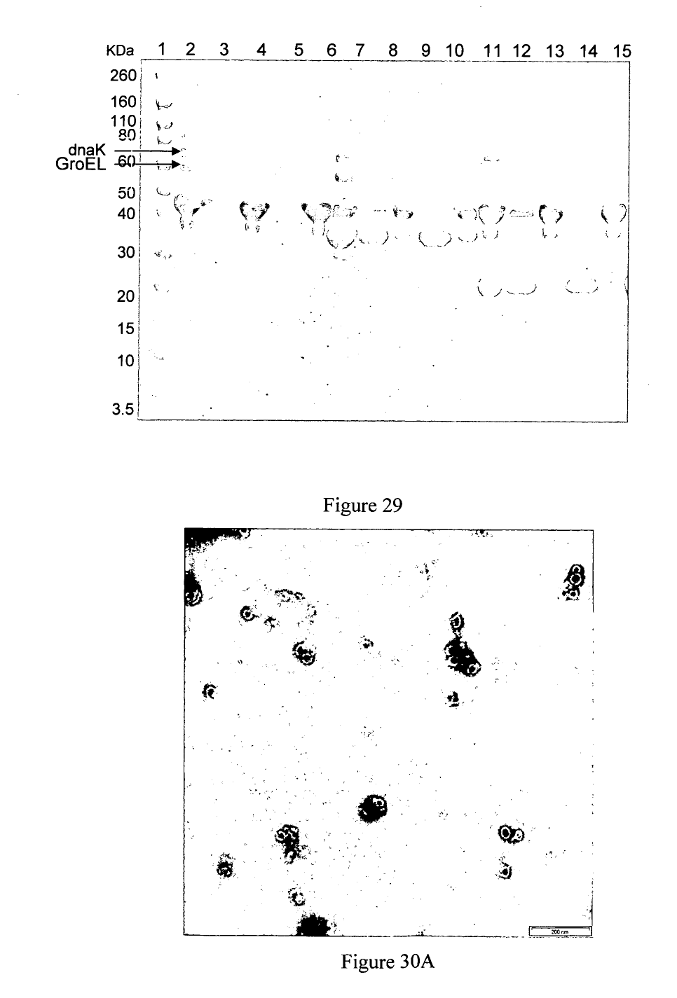

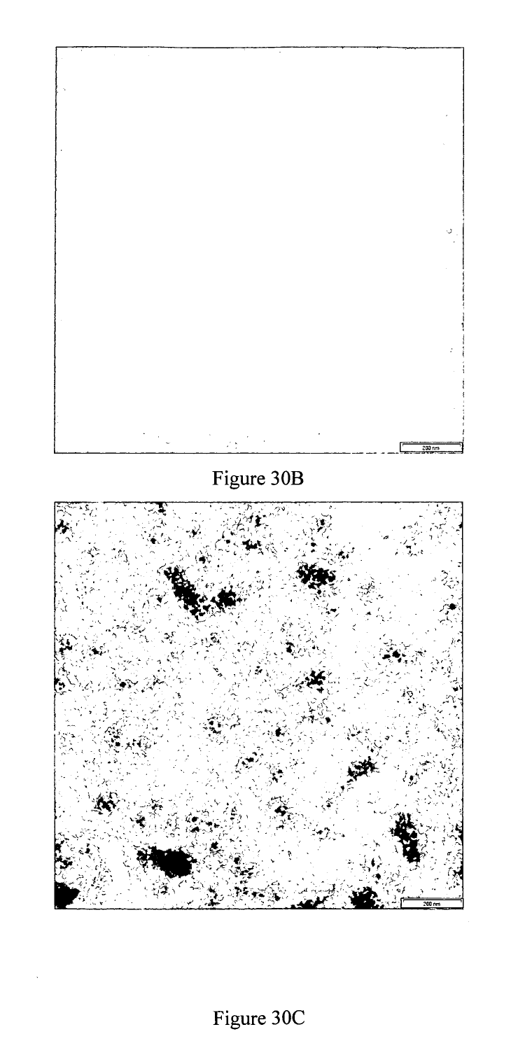

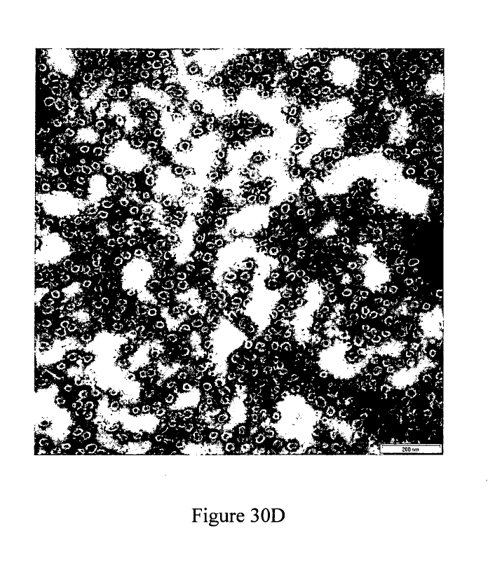

Treatment of impure VP1 samples with (NH₄)₂SO₄ at 15%, 20%. (FIGS. 29-30)

Two samples of VP1 capsomeres purified by size-exclusion chromatography (S200VP1), and a mixture of VP1 and GST obtained by thrombin treatment of GST-purified fusion protein were centrifuged (15000 rpm, 4° C., 5 min). Supernatant comprising 2 aliquots of 100 μL and 1 aliquot of 40 μL (“Untreated Control”) was recovered for each sample. Saturated (NH₄)₂SO₄ solution was added to the 100 μL aliquots to obtain a final concentration of 15% and 20% (all % v/v of saturated (NH₄)₂SO₄). Mixtures were incubated on a roller mixer (4° C., 1 h) and then centrifuged (10000 rpm, 4° C., 10 min). Supernatant was collected and the precipitate was re-suspended in 100 μL L buffer. Samples before and after assembled were analysed by TEM and SDS-PAGE gel electrophoresis.

The two S200 purified VP1 samples (FIG. 29, Lane 2 and 7) showed very high levels of both GroEL and dnaK. Treatment of both with 15% (v/v) and 20% (v/v) (NH₄)₂SO₄ gave VP1 capsomeres having significantly reduced levels of these chaperonin contaminants. Removal of GroEL and dnaK from VP1 led to improved VLP homogeneity and reduced aggregate levels (FIG. 30, A and B). Treatment of a mixture of contaminated VP1 and GST with 15% (v/v) and 20% (v/v) (NH₄)₂SO₄ improved capsomere quality (FIG. 29, Lane 14 and 16) and hence improved VLP quality (FIG. 30, C and D).

### REFERENCES

Chuan, Y. P., Lua, L. H. L. & Middelberg, A. P. J. 2008a. High-level expression of soluble viral structural protein in Escherichia coli. J. Biotechnol. 134, 64-71.

Chuan, Y. P., Fan, Y. Y., Lua, L. & Middelberg, A. P. J. 2008b Quantitative analysis of virus-like particle size and distribution by field-flow fractionation. Biotechnol. Bioeng. 99, 1425-1433.

Lipin, D. I., Lua, L. H. L. & Middelberg, A. P. J. 2008. Quaternary size distribution of soluble aggregates of glutathione-S-transferase-purified viral protein as determined by asymmetrical flow field flow fractionation and dynamic light scattering. J. Chromatogr. A 1190, 204-214.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

TABLE 1
Protein Summary Report and Index for Hsp 60 Mass Spec results

| | Accession | Mass | Score | Description |
|---|---|---|---|---|
| 1. | Q6UDB4 | 57268 | 99 | tr\|Q6UDB4\|60 kDa chaperonin (Fragment).[Escherichia coli] |
| 2. | Q6UDB0 | 57263 | 97 | tr\|Q6UDB0\|60 kDa chaperonin (Fragment).[Escherichia coli] |
| 3. | P0A6F6 | 57293 | 96 | sp\|P0A6F6\|60 kDa chaperonin (Protein Cpn60) (groEL protein).[Escherichia coli O6] |
| 4. | P0A6F7 | 57293 | 96 | sp\|P0A6F7\|60 kDa chaperonin (Protein Cpn60) (groEL protein).[Escherichia coli O157:H7] |
| 5. | P0A6F5 | 57293 | 96 | sp\|P0A6F5\|60 kDa chaperonin (Protein Cpn60) (groEL protein).[Escherichia coli] |
| 6. | P0A6F8 | 57293 | 96 | sp\|P0A6F8\|60 kDa chaperonin (Protein Cpn60) (groEL protein).[Shigella flexneri] |

TABLE 2
Results List

| 1. | Q6UDB4 Mass: 57268 Score: 99 Expect: 0.00096 Queries matched: 19 | | | | | | | | | |
|---|---|---|---|---|---|---|---|---|---|---|
| | tr\|Q6UDB4\|60 kDa chaperonin (Fragment). [Escherichia coli] | | | | | | | | | |
| | Observed | Mr (expt) | Mr (calc) | Delta | Start | | End | Miss | Peptide | |
| | 803.4064 | 802.3991 | 802.4007 | −0.0016 | 446 | — | 452 | 0 | R.AMEAPLR.Q + Oxidation (M) | |
| | | | | | | | | | (SEQ ID NO: 2) | |
| | 875.4230 | 874.4157 | 874.4283 | −0.0126 | 59 | — | | 65 | 0 | R.EIELEDK.F |
| | | | | | | | | | | (SEQ ID NO: 3) |
| | 985.5951 | 984.5878 | 984.5604 | 0.0274 | 19 | — | | 28 | 0 | R.GVNVLADAVK.V |
| | | | | | | | | | | (SEQ ID NO: 4) |
| | 1011.4684 | 1010.4611 | 1010.5145 | −0.0534 | 396 | — | | 404 | 0 | R.VEDALHATR.A |
| | | | | | | | | | | (SEQ ID NO: 5) |
| | 1201.3792 | 1200.3719 | 1200.5735 | −0.2015 | 431 | — | | 441 | 0 | R.GQNEDQNVGIK.V |
| | | | | | | | | | | (SEQ ID NO: 6) |
| | 1215.6483 | 1214.6410 | 1214.6580 | −0.0170 | 232 | — | | 242 | 9 | R.EMLPVLEAVAK.A + Oxidation (M) |
| | | | | | | | | | | (SEQ ID NO: 7) |
| | 1454.6401 | 1453.6328 | 1453.6321 | 0.0007 | 351 | — | | 362 | 0 | R.QQIEEATSDYDR.E |
| | | | | | | | | | | (SEQ ID NO: 8) |
| | 1567.8368 | 1566.8295 | 1566.8730 | −0.0434 | 405 | — | | 421 | 0 | R.AAVEEGVVAGGGVALIR.V |
| | | | | | | | | | | (SEQ ID NO: 9) |
| | 1711.7782 | 1710.7709 | 1710.7696 | 0.0013 | 351 | — | | 364 | 1 | R.QQIEEATSDYDREK.L |
| | | | | | | | | | | (SEQ ID NO: 10) |
| | 1845.8000 | 1844.7927 | 1844.9116 | −0.1189 | 328 | — | | 345 | 0 | K.DTTTIIDGVGEEAAIQGR.V |
| | | | | | | | | | | (SEQ ID NO: 11) |
| | 1845.8564 | 1844.8491 | 1844.9116 | −0.0625 | 328 | — | | 345 | 0 | K.DTTTIIDGVGEEAAIQGR.V |
| | | | | | | | | | | (SEQ ID NO: 12) |
| | 1956.9117 | 1955.9044 | 1955.9986 | −0.0942 | 453 | — | | 470 | 0 | R.QIVLNCGEEPSVVANTVK.G + |
| | | | | | | | | | | Carbamidomethyl (C) |
| | | | | | | | | | | (SEQ ID NO: 13) |
| | 2042.8247 | 2041.8174 | 2041.9336 | −0.1162 | 59 | — | | 75 | 1 | R.EIELEDKFENMGAQMVK.E + 2 Oxidation (M) |
| | | | | | | | | | | (SEQ ID NO: 14) |
| | 2402.1068 | 2401.0995 | 2401.2336 | −0.1341 | 81 | — | | 105 | 0 | K.ANDAAGDGTTTATVLAQAIITEGLK.A |
| | | | | | | | | | | (SEQ ID NO: 15) |
| | 2614.2553 | 2613.2480 | 2613.3134 | −0.0654 | 169 | — | | 193 | 1 | K.VGKEGVITVEDGTGLQDELDVVEGR.Q |
| | | | | | | | | | | (SEQ ID NO: 16) |
| | 2739.3520 | 2738.3447 | 2738.4888 | −0.1441 | 243 | — | | 268 | 0 | K.AGKPLLIIAEDVEGEALATLVVNTMR.G + |
| | | | | | | | | | | Oxidation (M) |
| | | | | | | | | | | (SEQ ID NO: 17) |
| | 2998.1050 | 2997.0977 | 2997.2644 | −0.1667 | 471 | — | | 498 | 0 | K.GGDGNYGYNAATEEYGNMIDMGILDPTK.V + |
| | | | | | | | | | | 2 Oxidation (M) |
| | | | | | | | | | | (SEQ ID NO: 18) |
| | 3111.3376 | 3110.3303 | 3110.6216 | −0.2912 | 198 | — | | 225 | 0 | R.GYLSPYFINKPETGAVELESPFILLADK.K |
| | | | | | | | | | | (SEQ ID NO: 20) |
| | 3239.5341 | 3238.5268 | 3238.7165 | −0.1897 | 198 | — | | 226 | 1 | R.GYLSPYFINKPETGAVELESPFILLADKK.I |
| | | | | | | | | | | (SEQ ID NO: 21) |

No match to: 1029.5460, 1086.6507, 1183.4973, 1301.7874, 1315.6841, 1408.6806, 1437.6904, 1439.7989, 1479.7721,
1520.6835, 1532.7008, 1624.8414, 1662.8336, 1694.7565, 1697.8815, 1719.8643, 1754.8587, 1764.7046, 1771.4027,
2269.0379, 2326.0054, 2356.9208, 2431.1537, 2457.1748, 2483.1135, 2513.1840, 2559.2552, 2675.2313, 2756.3648,
2796.3554, 2867.1800, 2883.1741, 2923.1902, 3112.4572, 3212.4704, 3269.4149, 3296.6671

TABLE 3
Protein Summary Report and Index for dnaK Mass Spec results

| | Accession | Mass | Score | Description |
|---|---|---|---|---|
| 1. | A7ZHA4 | 69130 | 248 | sp\|A7ZHA4\|Chaperone protein dnaK Tax_Id = 331111 [Escherichia coli O139:H28] |
| 2. | P0A6Z0 | 69130 | 248 | sp\|P0A6Z0\|Chaperone protein dnaK Tax_Id = 83334 [Escherichia coli O157:H7] |
| 3. | A7ZVV7 | 69146 | 248 | sp\|A7ZVV7\|Chaperone protein dnaK Tax_Id = 331112 [Escherichia coli O9:H4] |
| 4. | A1A766 | 69130 | 248 | sp\|A1A766\|Chaperone protein dnaK Tax_Id = 405955 [Escherichia coli O1:K1/APEC] |
| 5. | Q0TLX5 | 69130 | 248 | sp\|Q0TLX5\|Chaperone protein dnaK Tax_Id = 362663 [Escherichia coli O6:K15:H31] |
| 6. | P0A6Y9 | 69130 | 248 | sp\|P0A6Y9\|Chaperone protein dnaK Tax_Id = 217992 [Escherichia coli O6] |
| 7. | B1IRG0 | 69130 | 248 | sp\|B1IRG0\|Chaperone protein dnaK Tax_Id = 481805 [Escherichia coli] |
| 8. | P0A6Y8 | 69130 | 248 | sp\|P0A6Y8\|Chaperone protein dnaK Tax_Id = 83333 [Escherichia coli] |
| 9. | Q1RGI8 | 69130 | 248 | sp\|Q1RGI8\|Chaperone protein dnaK Tax_Id = 364106 [Escherichia coli] |
| 10. | Q326K7 | 69130 | 248 | sp\|Q326K7\|Chaperone protein dnaK Tax_Id = 300268 [Shigella boydii serotype 4] |

TABLE 4
Results List for dnaK

| 1. | A7ZHA4 Mass: 69130 Score: 248 Expect: 1.7e-018 Queries matched: 33 | | | | | | | | |
|---|---|---|---|---|---|---|---|---|---|
| | sp\|A7ZHA4\|Chaperone protein dnaK Tax_Id = 331111 [Escherichia coli O139:H28] | | | | | | | | |
| | Observed | Mr (expt) | Mr (calc) | Delta | Start | | End | Miss | Peptide |
| | 816.4480 | 815.4407 | 815.4137 | 0.0270 | 247 | — | 253 | 0 | K.DQGIDLR.N |
| | | | | | | | | | (SEQ ID NO: 222) |
| | 885.5660 | 884.5587 | 884.5443 | 0.0144 | 160 | — | 167 | 1 | R.IAGLEVKR.I |
| | | | | | | | | | (SEQ ID NO: 23) |
| | 960.4850 | 959.4777 | 959.4494 | 0.0283 | 254 | — | 261 | 0 | R.NDPLAMQR.L + Oxidation (M) |
| | | | | | | | | | (SEQ ID NO: 24) |
| | 1002.5270 | 1001.5197 | 1001.4778 | 0.0420 | 26 | — | 34 | 0 | R.VLENAEGDR.T |
| | | | | | | | | | (SEQ ID NO: 25) |
| | 1021.5650 | 1020.5577 | 1020.5240 | 0.0337 | 529 | — | 536 | 0 | K.FEELVQTR.N |
| | | | | | | | | | (SEQ ID NO: 26) |
| | 1050.5340 | 1049.5267 | 1049.4778 | 0.0489 | 77 | — | 84 | 0 | R.FQDEEVQR.D |
| | | | | | | | | | (SEQ ID NO: 27) |
| | 1149.6820 | 1148.6747 | 1148.6190 | 0.0558 | 528 | — | 536 | 1 | R.KFEELVQTR.N |
| | | | | | | | | | (SEQ ID NO: 28) |
| | 1179.6780 | 1178.6707 | 1178.6084 | 0.0623 | 353 | — | 362 | 1 | K.VAEFFGKEPR.K |
| | | | | | | | | | (SEQ ID NO: 29) |
| | 1206.6580 | 1205.6507 | 1205.5789 | 0.0718 | 76 | — | 84 | 1 | R.RFQDEEVQR.D |
| | | | | | | | | | (SEQ ID NO: 30) |
| | 1277.7160 | 1276.7087 | 1276.6272 | 0.0815 | 537 | — | 547 | 0 | R.NQGDHLLHSTR.K |
| | | | | | | | | | (SEQ ID NO: 31) |
| | 1286.7760 | 1285.7687 | 1285.6878 | 0.0810 | 305 | — | 315 | 0 | K.LESLVEDLVNR.S |
| | | | | | | | | | (SEQ ID NO: 32) |
| | 1290.6930 | 1289.6857 | 1289.6099 | 0.0758 | 503 | — | 514 | 0 | K.ASSGLNEDEIQK.M |
| | | | | | | | | | (SEQ ID NO: 33) |
| | 1307.7950 | 1306.7877 | 1306.7034 | 0.0844 | 353 | — | 363 | 2 | K.VAEFFGKEPRK.D |
| | | | | | | | | | (SEQ ID NO: 34) |
| | 1395.8750 | 1394.8677 | 1394.7809 | 0.0868 | 236 | — | 246 | 1 | R.LINYLVEEFKK.D |
| | | | | | | | | | (SEQ ID NO: 35) |
| | 1405.8130 | 1404.8057 | 1404.7222 | 0.0835 | 537 | — | 548 | 1 | R.NQGDHLLHSTRK.Q |
| | | | | | | | | | (SEQ ID NO: 36) |
| | 1427.8840 | 1426.8767 | 1426.7854 | 0.0914 | 110 | — | 122 | 1 | K.MAPPQISAEVLKK.M + Oxidation (M) |
| | | | | | | | | | (SEQ ID NO: 37) |
| | 1485.9320 | 1484.9247 | 1484.8198 | 0.1049 | 303 | — | 315 | 1 | R.AKLESLVEDLVNR.S |
| | | | | | | | | | (SEQ ID NO: 38) |
| | 1500.8680 | 1499.8607 | 1499.7620 | 0.0987 | 93 | — | 106 | 0 | K.IIAADNGDAWVEVK.G |
| | | | | | | | | | (SEQ ID NO: 39) |
| | 1544.9460 | 1543.9387 | 1543.8358 | 0.1029 | 57 | — | 70 | 0 | R.QAVTNPQNTLFAIK.R |
| | | | | | | | | | (SEQ ID NO: 40) |
| | 1598.9460 | 1597.9387 | 1597.8212 | 0.1175 | 453 | — | 467 | 0 | K.SLGQFNLDGINPAPR.G |
| | | | | | | | | | (SEQ ID NO: 41) |
| | 1660.0120 | 1659.0047 | 1658.8879 | 0.1168 | 168 | — | 183 | 0 | R.IINEPTAAALAYGLDK.G |
| | | | | | | | | | (SEQ ID NO: 42) |
| | 1701.0780 | 1700.0707 | 1699.9369 | 0.1338 | 57 | — | 71 | 1 | R.QAVTNPQNTLFAIKR.L |
| | | | | | | | | | (SEQ ID NO: 43) |
| | 1816.1330 | 1815.1257 | 1814.9890 | 0.1367 | 167 | — | 183 | 1 | K.RIINEPTAAALAYGLDK.G |
| | | | | | | | | | (SEQ ID NO: 44) |
| | 2192.2530 | 2191.2457 | 2191.0505 | 0.1952 | 518 | — | 536 | 2 | R.DAEANAEADRKFEELVQTR.N |
| | | | | | | | | | (SEQ ID NO: 45) |
| | 2321.3400 | 2320.3327 | 2320.1403 | 0.1924 | 4 | — | 25 | 0 | K.IIGIDLGTTNSCVAIMDGTTPR.V + Oxidation (M) |
| | | | | | | | | | (SEQ ID NO: 46) |
| | 2346.4490 | 2345.4417 | 2345.2227 | 0.2190 | 35 | — | 56 | 1 | R.TTPSIIAYTQDGETLVGQPAKR.Q |
| | | | | | | | | | (SEQ ID NO: 47) |
| | 2372.4020 | 2371.3947 | 2371.1730 | 0.2218 | 468 | — | 489 | 0 | R.GMPQIEVTFDIDADGILHVSAK.D + Oxidation (M) |
| | | | | | | | | | (SEQ ID NO: 48) |
| | 2423.5020 | 2422.4947 | 2422.2704 | 0.2243 | 363 | — | 387 | 1 | R.KDVNPDEAVAIGAAVQGGVLTGDVK.D |
| | | | | | | | | | (SEQ ID NO: 49) |
| | 2441.5170 | 2440.5097 | 2440.2810 | 0.2288 | 322 | — | 345 | 0 | K.VALQDAGLSVSDIDDVILVGGQTR.M |
| | | | | | | | | | (SEQ ID NO: 50) |
| | 2532.5300 | 2531.5227 | 2531.2755 | 0.2472 | 271 | — | 294 | 0 | K.IELSSAQQTDVNLPYITADATGPK.H |
| | | | | | | | | | (SEQ ID NO: 51) |
| | 2653.5500 | 2652.5427 | 2652.2921 | 0.2506 | 1 | — | 25 | 1 | -.MGKIIGIDLGTTNSCVAIMDGTTPR.V + |
| | | | | | | | | | 2 Oxidation (M) |
| | | | | | | | | | (SEQ ID NO: 52) |
| | 2731.6760 | 2730.6687 | 2730.4076 | 0.2612 | 269 | — | 294 | 1 | K.AKIELSSAQQTDVNLPYITADATGPK.H |
| | | | | | | | | | (SEQ ID NO: 53) |
| | 2869.6650 | 2868.6577 | 2868.3818 | 0.2760 | 126 | — | 151 | 0 | K.TAEDYLGEPVTEAVITVPAYFNDAQR.Q |
| | | | | | | | | | (SEQ ID NO: 54) |

No match to: 804.2990, 823.1120, 842.5330, 870.5500, 1045.5990, 1126.5790, 1527.9160, 1566.8640, 1684.0260,
1930.0470, 1941.0870, 2211.2930, 2269.2510, 2282.2370

1. A method of preparing an isolated virus particle and/or virus-like particle (VLP), wherein said method includes the step of contacting a mixture comprising an isolated virus particle and/or VLP with a polymer, a salt or an acid at a concentration such that the isolated virus particle and/or VLP is selectively separated in an aqueous phase.

2. The method of claim 1, wherein a correctly-formed isolated virus particle and/or correctly-formed VLP is selectively separated in an aqueous phase.

3. The method of claim 1, wherein the salt is selected from ammonium sulphate and sodium chloride.

4. The method of claim 3, wherein the salt is ammonium sulphate.

5. The method of claim 4, wherein the ammonium sulphate is present at a final concentration of between about 20% v/v and about 35% v/v.

6-8. (canceled)

9. The method of claim 1, wherein the polymer is selected from a polyethylene (PEG) and a polyelectrolyte.

10. The method of claim 9, wherein the PEG is present at a final concentration of between about 1.5% w/v and about 5% w/v.

11. The method of claim 9, wherein the PEG is present at a final concentration of between about 1.5% w/v and about 5% w/v.

12. The method of claim 1, wherein the VLP is selected from a polyomavirus VLP and a human papillomavirus VLP.

13. The method of claim 1, wherein the VLP is selected from a polyomavirus VLP and a human papillomavirus VLP.

14. The method of claim 1, wherein the VLP comprises one or more immunogenic epitopes derived from a different pathogen.

15. The method of claim 1, wherein the different pathogen is selected from an influenza virus, Group A Streptococcus pyogenes and a Hendra virus.

16-26. (canceled)

27. A method of a preparing a capsomere substantially-free of one or more host cell derived chaperone proteins, said method including the step of contacting a mixture comprising a capsomere and at least one host cell derived chaperone protein with an agent such that the capsomere is selectively separated from at least one host cell derived chaperone protein to thereby prepare a capsomere substantially-free of one or more host cell derived chaperone proteins.

28. The method of claim 27, wherein the agent is selected from the group consisting of an anion exchanger chromatographic material, a cation exchanger chromatographic material, ammonium sulphate, a PEG and combinations thereof.

29. The method of claim 28, wherein the agent is ammonium sulphate.

30. The method of claim 28, wherein the agent is an anion exchanger chromatographic material and ammonium sulphate.

31. The method of claim 28, wherein the agent is a cation exchanger chromatographic material and ammonium sulphate.

32. (canceled)

33. The method of claim 29, wherein the final concentration of ammonium sulphate is between about 12.5% v/v and about 50% v/v.

34-36. (canceled)

37. The method of claim 28, wherein the agent is an anion exchanger chromatographic material and a cation exchanger chromatographic material.

38. The method of claim 37, wherein contact of the mixture with an anion exchanger chromatographic material precedes contact with a cation exchanger chromatographic material.

39-43. (canceled)

44. The method of claim 27, wherein the at least one host cell derived chaperone protein is a heat shock protein.

45. The method of claim 44, wherein the heat shock protein is selected from heat shock protein 60, heat shock protein 70, GroEL and dnaK.

46. (canceled)

47. The method of claim 45, wherein when the chaperone is GroEL, the agent is an anion exchanger chromatographic material.

48. The method of claim 45, wherein when the at least one host cell derived chaperone protein is dnaK, the agent is a cationic exchanger chromatographic material.

49. The method of claim 27, wherein the capsomere is a derived from a polyomavirus structural protein.

50. The method of claim 49, wherein the polyomavirus structural protein is a murine polyomavirus protein.

51. The method of claim 49, wherein the polyomavirus structural protein is VP1.

52-63. (canceled)

64. The method of claim 9, wherein the polyelectrolyte is an anionic polyelectrolyte selected from the group consisting of polystyrenesulfonic acid, polyacrylic acid and polystyrenesulfonic acid.
